SEQUENCE LISTING (2 sequences)

SEQ ID NO: 1 - protein, 1273 aa, Homo sapiens
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 259 aa, synthetic construct
MFVFLVLLPLVSSQVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHHHHGLNDIFEAQKIEWHE

(19) United States
(12) Patent Application Publication
(10) Pub. No.: US 2024/0241109 A1
(43) Pub. Date: Jul. 18, 2024

(54) Methods to Identify Mutation Specific B Cells and Restore Therapeutic Antibody Efficacy Against Viral Variants

(71) Applicant: National Jewish Health, Denver, CO (US)

(72) Inventor: Haolin Liu, Denver, CO (US)

(21) Appl. No.: 18/405,350

(22) Filed: Jan. 5, 2024

Related U.S. Application Data
(60) Provisional application No. 63/478,540, filed on Jan. 5, 2023.

(51) Int. Cl. G01N 33/50

(52) U.S. Cl. CPC G01N 33/5091; G01N 33/5052; G01N 2333/11; G01N 2333/165

(57) ABSTRACT

Disclosed herein are methods of restoring therapeutic antibody efficacy against a viral variant, of identifying wild-type specific memory B cells, cross-reactive memory B cells and mutation specific memory B cells in a subject following viral vaccination, and of monitoring a subject's memory B cell response against a vaccine antigen and/or a viral variant thereof.

### CROSS-REFERENCE TO RELATED APPLICATIONS

This application claims the benefit of priority under 35 U.S.C. § 119(e) to U.S. Provisional Patent Application No. 63/478,540, filed Jan. 5, 2023. The entire disclosure of U.S. Provisional Patent Application No. 63/478,540 is incorporated herein by reference.

### GOVERNMENT SUPPORT STATEMENT

This invention was made with government support under 5R01 GM135421, awarded by The National Institute of General Medical Sciences. The government has certain rights to the invention.

### REFERENCE TO A SEQUENCE LISTING

This application contains a Sequence Listing submitted electronically as an XML file. The XML file, named “2879-241.xml”, has a size of 4394 Bytes, and was recorded on Apr. 3, 2024. The information contained in the XML file is incorporated herein by reference in its entirety.

### BACKGROUND

Severe acute respiratory syndrome-related coronavirus 2 (SARS-COV-2) remains a tremendous threat to the public health globally (COVID-19 Incidence and Death Rates Among Unvaccinated and Fully Vaccinated Adults with and Without Booster Doses During Periods of Delta and Omicron Variant Emergence—25 U.S. Jurisdictions, Apr. 4-Dec. 25, 2021). To combat the virus, several therapeutic monoclonal antibodies (mAbs) have been approved for emergency use (Kaplon, H. & Reichert, J. M. Antibodies to watch in 2021. mAbs 13, 1860476, (2021)), however, newly emerging variants can manage to escape the binding by mAbs (Chen, R. E. et al. In vivo monoclonal antibody efficacy against SARS-COV-2 variant strains. Nature 596, 103-108, (2021)). For example, Bamlanivimab completely loses efficacy to the Beta, Gamma, Lambda, Mu and Omicron variants, and partially to Delta variant (Liu, H., et al. SARS-COV-2 Variants of Concern and Variants of Interest Receptor Binding Domain Mutations and Virus Infectivity. Frontiers in Immunology 13, (2022)). Meanwhile, different types of vaccines have also been approved to immunize the public⁴⁻⁷ (Polack, F. P. et al. Safety and Efficacy of the BNT162b2 mRNA Covid-19 Vaccine. N. Engl. J. Med. 383, 2603-2615, (2020); Baden, L. R. et al. Efficacy and Safety of the mRNA-1273 SARS-COV-2 Vaccine. N Engl J Med, (2020); Falsey, A. R. et al. Phase 3 Safety and Efficacy of AZD1222 (ChAdOx1 nCOV-19) Covid-19 Vaccine. New England Journal of Medicine, (2021); and Gao, Q. et al. Development of an inactivated vaccine candidate for SARS-COV-2. Science 369, 77-81, (2020)), and again, the most recent surge of Omicron variant, the BA1 version of which had 32 amino acids mutations in the spike region (Liu, H., et al., 2022), could evade more than 80% of serum antibody binding (Cao, Y. et al. Omicron escapes the majority of existing SARS-COV-2 neutralizing antibodies. Nature 602, 657-663, (2022)). Concerns over whether the current wildtype spike-based vaccine could still work effectively against the variants are rising.

An antibody recognizes an antigen by epitope. An epitope is formed by multiple amino acids, ranging from couple to 20s, from the antigen (Bosshard, H. R. in Peptides (ed Bernd Gutte) 419-454 (Academic Press, 1995)). The mutation from the virus variant can reduce or abolish mAb binding only by falling right onto the epitope (Liu, H. et al. 501Y.V2 and 501Y.V3 variants of SARS-COV-2 lose binding to bamlanivimab in vitro. mAbs 13, 1919285, (2021)). The receptor binding domain (RBD) contains around 220 amino acids and is the major antigenic site of the full length spike, counting for 90% serum antibody induced by SARS-COV-2 virus infection or vaccination (Piccoli, L. et al. Mapping Neutralizing and Immunodominant Sites on the SARS-COV-2 Spike Receptor-Binding Domain by Structure-Guided High-Resolution Serology. Cell 183, 1024-1042. (2020)). The RBD contains four classes of antibody binding hotspots based on the conformational structure of RBD (Barnes, C. O. et al. SARS-CoV-2 neutralizing antibody structures inform therapeutic strategies. Nature 588, 682-687, (2020)). Although the Omicron BA1 variant has 15 amino acid mutations spanning the four hotspots of RBD domain, the cross reactivity of both antibody and B cells to Omicron RBD induced by the wildtype spike based vaccine has been reported (Goel, R. R. et al. Efficient recall of Omicron-reactive B cell memory after a third dose of SARS-COV-2 mRNA vaccine. Cell, (2022)), as more amino acids stay the same between BA1 and wildtype RBD.

Serum antibody is secreted by plasma B cells, most of which have been selected in the germinal center to secrete high-affinity antibody against the original antigen (Viant, C. et al. Antibody Affinity Shapes the Choice between Memory and Germinal Center B Cell Fates. Cell 183, 1298-1311. (2020)). Through mutating, the virus variants could escape the polyclonal serum antibodies binding (Piccoli, L. et al. 2020). When an amino acid mutates in a virus variant, it may also become a new epitope. However, little is known about whether the current COVID-19 vaccines could induce B cells specifically that recognize the mutated amino acid(s).

The current mAb drugs and vaccines have been developed based on the spike sequence of the ancestral strain (Wildtype). However, due to the low fidelity of RNA polymerases in RNA virus (Peck Kayla, M., et al. Complexities of Viral Mutation Rates. Journal of Virology 92. 2018), SARS-COV-2 virus variants with mutations in the spike region have emerged and dominate infections at various times. Some mutations not only enhance virus binding affinity to angiotensin-converting enzyme-2 (ACE2), but also reduce or even abolish the efficacy of mAbs and serum antibodies induced by vaccines (Greaney, A. J. et al. Complete Mapping of Mutations to the SARS-COV-2 Spike Receptor-Binding Domain that Escape Antibody Recognition. Cell Host & Microbe 29, 44-57. (2021)). For example, the a surge of Omicron BA1 variant with 32 amino acid mutations in the spike region, led to a reduction of 80% of serum antibody binding (Cao, Y. et al. B.1.1.529 escapes the majority of SARS-COV-2 neutralizing antibodies of diverse epitopes. bioRxiv, 2021.2012.2007.470392, (2021)).

Most current research focuses on serum antibody and virus neutralization. Antibodies are secreted by plasma cells, the majority of which are short-lived (Nutt, S. L., Hodgkin, P. D., Tarlinton, D. M. & Corcoran, L. M. The generation of antibody-secreting plasma cells. Nature Reviews Immunology 15, 160-171, (2015)). This appears to be true for SARS-COV-2 infection and vaccines since serum antibody in these cases has a relatively short half life (<8 month). However, antibodies are not the only contributions of vaccines to B cell immunity. Infection and vaccination induce also the appearance of memory B cells that have a longer half-life than serum antibodies, perhaps because of higher expression of transcription factors, such as Bcl-2 (Nuñez, G., Hockenbery, D., McDonnell, T. J., Sorensen, C. M. & Korsmeyer, S. J. Bcl-2 maintains B cell memory. Nature 353, 71-73, (1991)). Upon antigen re-exposure either through virus infection or vaccine boosts, the memory B cells would proliferate and differentiate into antibody secreting B cells without further germinal center selection (Inoue, T., Shinnakasu, R. & Kurosaki, T. Generation of High Quality Memory B Cells. Frontiers in Immunology 12, (2022)). Therefore, repeat vaccination based on falling serum antibody level is misleading.

Besides longevity, it has been shown that memory B cells generated in mice also possess the ability to recognize specific antigen variants and not, necessarily, the immunizing antigen (Purtha, W. E., Tedder, T. F., Johnson, S., Bhattacharya, D. & Diamond, M. S. Memory B cells, but not long-lived plasma cells, possess antigen specificities for viral escape mutants. Journal of Experimental Medicine 208, 2599-2606, (2011)). However, little is known about the possibility that Spike vaccination induces memory B cells specific for Spike variants, but not the vaccinating Spike in humans. Therefore, there remains a need for new vaccine designs that understand the properties of variant specific B cells induced by the wildtype antigen.

### SUMMARY

One embodiment of the invention relates to a method of identifying wild-type specific memory B cells, cross-reactive memory B cells and mutation specific memory B cells in a subject, wherein the subject has been administered one or more doses of a COVID-19 vaccine, the method comprising: contacting a peripheral blood mononuclear cell (PBMC) sample from the subject with an equal concentration of: wild-type SARS-COV-2 RBD tetramers comprising a double colored fluorescent label, and a variant SARS-COV-2 RBD tetramer comprising a third color fluorescent label to identify wildtype specific memory B cells and cross-reactive memory B cells; and contacting the PBMC sample with an equal concentration of: variant SARS-COV-2 RBD tetramers comprising a double-colored fluorescent label, and a wild-type RBD tetramer comprising a third color fluorescent label to identify mutation specific memory B cells and cross-reactive memory B cells.

One embodiment of the invention relates to a method of determining the efficacy of a vaccine to a viral variant comprising: obtaining a peripheral blood mononuclear cell (PBMC) sample from a subject who has been administered one or more doses of a viral vaccine; contacting the PBMC sample with one or more viral variant receptor binding domain (RBD) tetramers, wherein the tetramers are each labeled with a different fluorescent label, and further contacting the sample with one or more corresponding wild-type RBD tetramers, wherein the wild-type tetramers are labeled with fluorescent labels different from the RBD tetramer labels; and determining binding levels of the wild-type RBD specific, cross-reactive and viral variant RBD specific memory B cells in the PBMC sample from the subject, wherein the levels of cross-reactive and viral variant specific memory B cells correlate with vaccine efficacy against the viral variant.

In one aspect, the step of contacting the PBMC sample from the subject comprises contacting the sample with an equal concentration of wild-type SARS-COV-2 RBD tetramers comprising a double-colored label, and a variant SARS-COV-2 RBD tetramer comprising a third color label.

One embodiment of the invention relates to a method of monitoring a subject's memory B cell response against a vaccine antigen and/or a viral variant thereof, the method comprising obtaining a PBMC sample from the subject prior to administration of a viral vaccine; obtaining a PBMC sample from the subject following administration of the viral vaccine administered; contacting the PBMC samples with wild-type double colored fluorescent labeled vaccine antigen tetramers and double colored variant antigen tetramers fluorescent labeled with third and fourth colors; detecting binding levels of wild-type specific, cross-reactive and variant antigen specific B cells in the PBMC samples; and comparing the levels of the wild-type specific, cross-reactive and variant antigen specific binding B cells in the sample following administration of the vaccine to the binding levels of memory B cells in the sample prior to administration of the vaccine; wherein percent changes of antigen binding B cell indicates the subject's B cell response to vaccination.

One embodiment of the invention relates to a method of restoring therapeutic antibody efficacy against a viral variant, comprising structurally modifying a non-variant/wild-type viral antibody to produce a viral variant specific antibody, the method comprising modifying one or more amino acids in the non-variant/wild-type antibody to be compatible with known mutations in the viral variant; measuring the binding affinity between the non-variant virus antibody and the variant viral protein, measuring the binding affinity between the mutation specific antibody and the variant viral protein, wherein similar binding affinities indicate the viral variant specific antibody has therapeutic efficacy.

In one aspect of any of the methods, the vaccine is a COVID-19 vaccine.

In one aspect of any of the methods, the viral variant is a SARS-COV-2 variant.

In yet another aspect of any of the methods, the vaccine is an influenza vaccine.

In still another aspect of any of the methods, the viral variant is an influenza variant.

In one aspect of any of the methods, the wild-type RBD tetramer is a wild-type SARS-COV-2 tetramer.

In yet another aspect of any of the methods, the variant RBD tetramer is a variant SARS-COV-2 tetramer.

### BRIEF DESCRIPTION OF THE DRAWINGS

FIGS. 1A-1H show that a single amino acid change can restore the efficacy of a monoclonal antibody against the mutation in the variant, making it a mutation specific antibody. Etesevimab (LY-CoV016) is a human therapeutic antibody targeting the receptor binding domain (RBD) of SARS-COV-2 spike protein. The affinity between Etesevimab and wildtype RBD is 6.8 nM measured by the surface plasmon resonance (SPR) with Biacore (FIG. 1A). The efficacy of Etesevimab is affected by the mutations at RBD 417 lysine (K417) position as lysine forms two salt bridges with aspartic acid 104 (D104) from the antibody heavy chain (VH) (FIG. 1E). RBD K417D mutation reduced Etesevimab affinity by 10-fold (FIG. 1B) due to loss of salt bridge interactions and adding weak negative charge repulsion at 5.6 angstrom between RBD D417 and VH D104 side chains (FIG. 1F). VH D104K mutant version of Etesevimab (K104-Etesevimab) does not bind wildtype RBD (FIG. 1C) as the strong positive charge repulsion at 4.0 angstrom between RBD K417 and VH K104 (FIG. 1G). However, K104-Etesevimab binds D417-RBD at 5.12 nM (FIG. 1D) as VH K104 restored two salt bridges with RBD D417 (FIG. 1H). Therefore, K104-Etesevimab is D417-RBD mutation specific antibody with only a single amino acid difference from Etesevimab.

FIG. 2 shows the specificity of double-colored wildtype RBD tetramer staining to identify RBD specific memory B cells from PBMCs of vaccinated donors (Gated on CD19+B cells).

FIGS. 3A and 3B: FIG. 3A shows the double-colored wildtype RBD tetramer and K484-RBD tetramer with a third color staining of PBMC to identify K484-RBD cross react and wildtype specific B cells. FIG. 3B shows the double colored K484-RBD tetramer and wildtype RBD tetramer with a third color staining of PBMC to identify K484 specific B cells and wildtype RBD cross-reactive B cells.

FIG. 4 shows SARS-COV-2 mRNA vaccine using the wildtype spike as the immunogen also induces B cells specifically binding SARS-COV-1 RBD, which has a 60 amino acid difference from SARS-COV-2 RBD.

FIGS. 5A and 5B show the stability of wildtype RBD (FIG. 5A) or Lambda RBD (FIG. 5B) memory B cells from same healthy donor at different time points after second dose of BNT162b2 mRNA vaccine.

FIGS. 6A and 6B show wildtype RBD (FIG. 6A) or Lambda RBD (FIG. 6B) binding B cell percentage from same donor before and after third dose of BNT162b2 mRNA vaccine.

FIGS. 7A and 7B shows the identification (FIG. 7A) and percentage change (FIG. 7B) of Lambda RBD mutation specific and cross-react B cell from same donor before and after third dose of BNT162b2 mRNA vaccination.

FIG. 8 shows ELISA measurement of serum RBD antibody after different types of COVID-19 vaccines. Bamlanvimab was used as standard.

FIG. 9 shows memory B cell level induced by Ad26.COV2.S adenovirus vaccine or BNT162b2 mRNA vaccine detected by double-colored RBD tetramer staining.

FIGS. 10A-10B show the overall conserved RBD backbone protein structure between SARS-COV family members. The overlay of Omicron BA1 and wildtype SARS-COV-2 RBD protein structure is shown in (FIG. 10A) and the overlay of SARS-COV-1 and SARS-COV-2 RBD is shown in (FIG. 10B).

FIGS. 11A-11D show Bamlaniviamb efficacy of blocking ACE2 interaction with RBD is reduced against Delta RBD and fully lost against Lambda RBD (FIG. 11A and FIG. 11B). This is also true for serum polyclonal antibodies induced by BNT162b2 vaccine. The Lambda RBD evades more Bamlanivimab and serum antibody binding from BNT162b2 mRNA vaccinated donors than Delta. (FIG. 11A) ELISA detection of Bamlanivimab binding to wild type, Delta or Lambda RBD coated plate. (FIG. 11B) ELISA detection of Bamlanivimab blocking efficacy of ACE2-mFc interaction with wild type, Delta or Lambda RBD. (FIG. 11C) BNT162b2 mRNA vaccinated sera from 10 donors were assayed on wild type, Delta or Lambda RBD coated ELISA plate to compare serum antibody evasion. The O.D. values of serum from the same donor on wild type, Delta or Lambda are linked with lines. (FIG. 11D) ELISA detection of serum antibody from 12 donors in blocking ACE2-mFc interaction with wild type, Delta or Lambda RBD. The symbols in FIGS. 11C and 11D indicate signal from wild type (circle), Delta (square) and Lambda (triangle) RBD respectively.

FIGS. 12A-12D show the distribution of 17 amino acid mutations of Omicron BA5 variant in the RBD region. (FIG. 12A) Top view. (FIG. 12B) Side view. The distribution of these 17 mutations in the four classes of antibody binding hot spots on RBD. (FIG. 12C) Front view of RBD for class 1 and class 3 antibody binding spots are in their respective circled areas. (FIG. 12D) Back view for class 2 and class 4 antibody binding spots are in their respective circled areas (see Frontiers in Immunology 13, (2022)).

FIGS. 13A and 13B show binding of SAR-COV-1 RBD after immunization with SARS-COV-2 spike. Staining of PBMC samples after wildtype spike vaccine. This staining is done by mixing four RBD tetramers at same concentration for staining, two with SARS-COV-2 wildtype RBD with PE and BV421 and two tetramers with SARS-COV-1 RBD with BV711 and APC. (FIG. 13A) Gating of wildtype RBD binding B cells. (FIG. 13B) Binding of SARS-COV-1 RBD by wildtype RBD binding B cells. This divides wildtype RBD binding B cells into SARS-CoV-1 RBD cross reactive and wildtype specific B cells.

FIGS. 14A and 14B show there are SARS-COV-1 RBD specific B cells after COVID-19 mRNA vaccination. Staining of PBMC samples after wildtype spike vaccine. This staining is done by mixing four RBD tetramers at same concentration for staining, two with SARS-COV-2 wildtype RBD with PE and BV421 and two tetramers with SARS-COV-1 RBD with BV711 and APC. (FIG. 14A) Gating of SARS-COV-1 RBD binding B cells. (FIG. 14B) Binding of wildtype RBD by SARS-COV-1 binding B cells. This divides SARS-COV-1 RBD binding B cells into SARS-COV-2 wildtype RBD cross reactive and SARS-COV-1 mutation specific B cells.

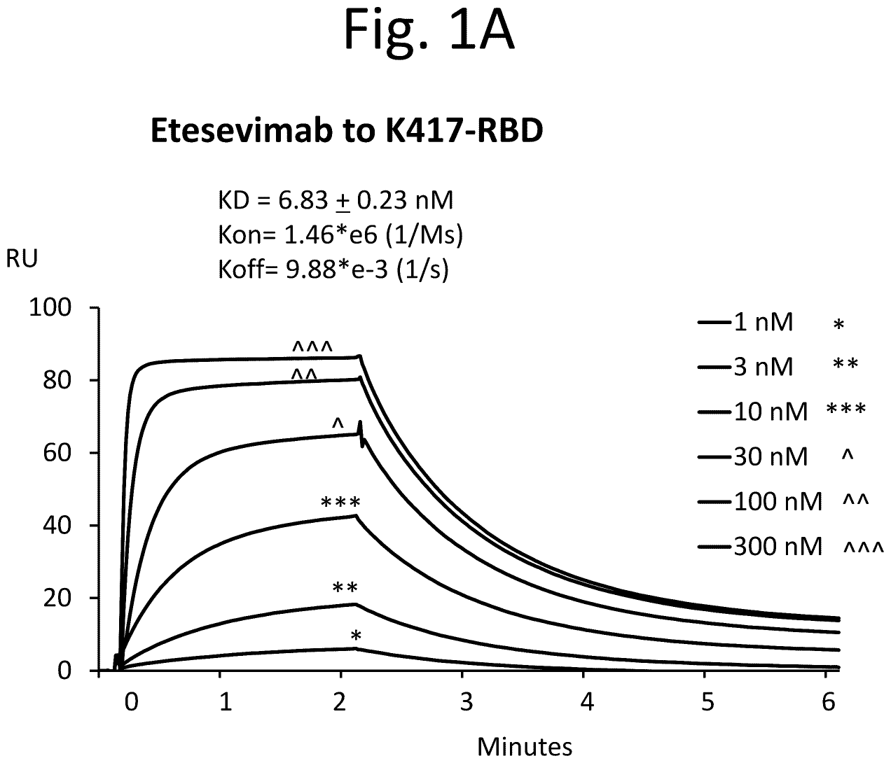

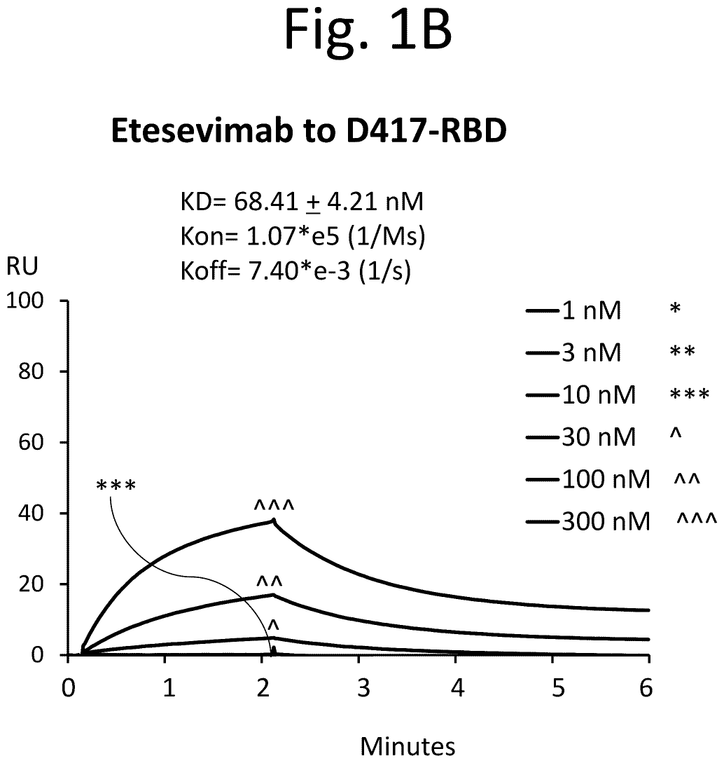

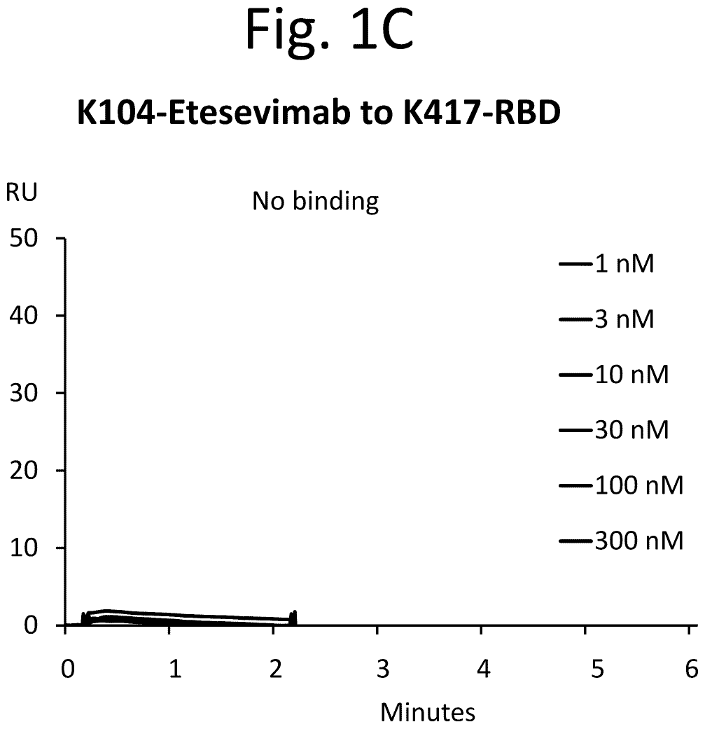

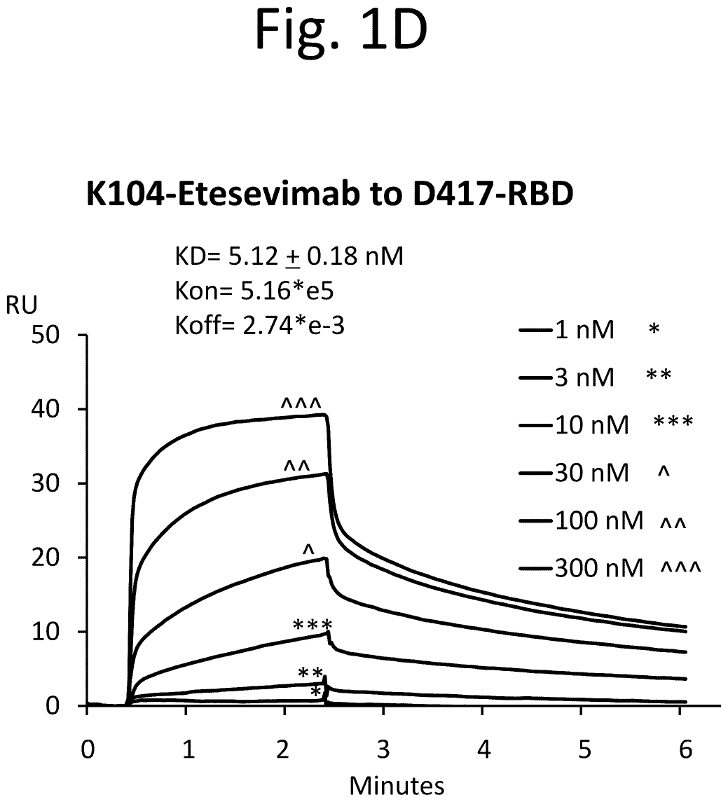

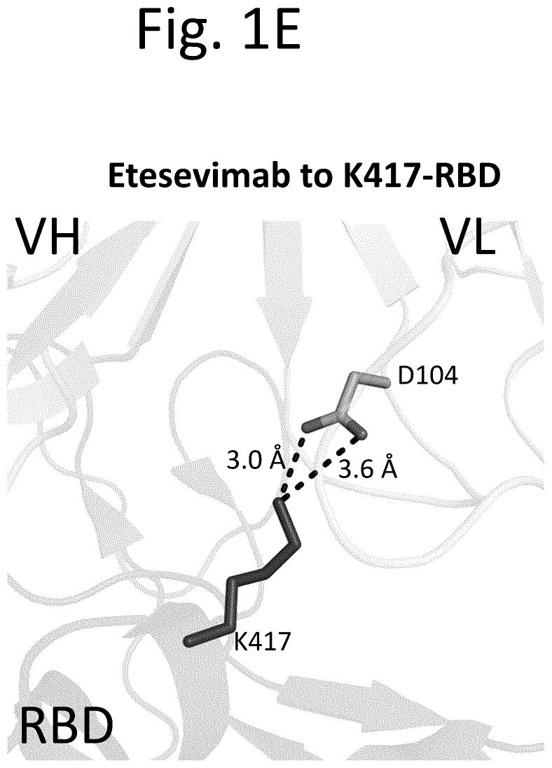

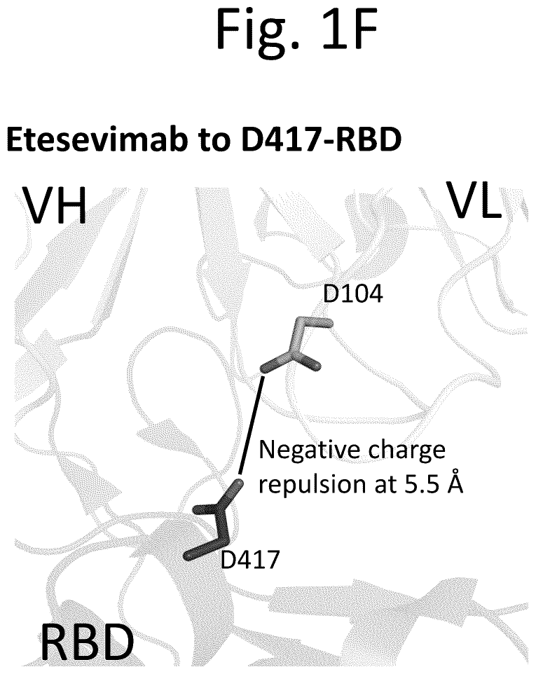

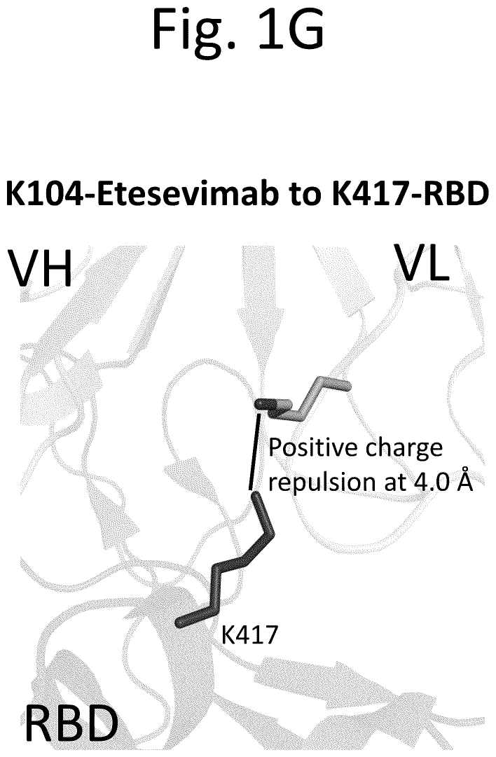

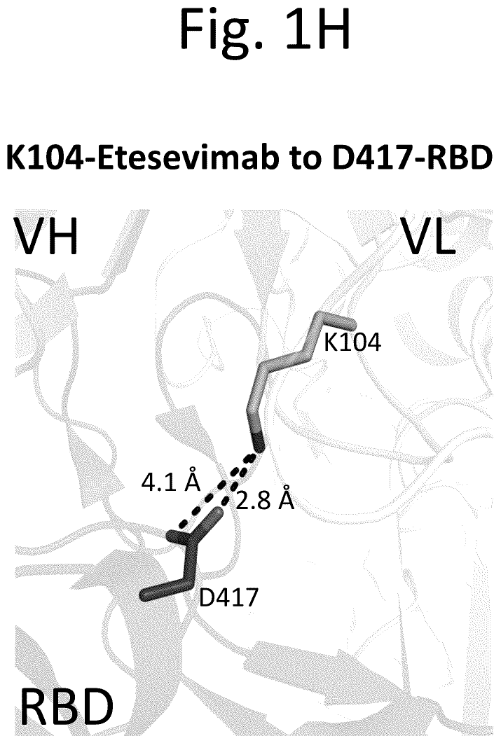

### DETAILED DESCRIPTION

The emergence and dominance of SARS-COV-2 variants with mutations in the spike region brings concern over the efficacy of therapeutic mAbs and vaccines as both of them are developed based on the wildtype spike sequence. mAbs lost efficacy against certain virus variants with mutations on their epitope. Moreover, virus variants also reduced serum antibody neutralization, especially true for the Omicron variants with the most number of mutations in spike so far. This is fully expected as serum antibody is secreted by plasma B cells which is selected to secrete high affinity antibody against the wildtype spike. However, memory B cells possess the antigen diversity against the mutations in mice. These cells are generated during the germinal center selection and preserved along with the wildtype antigen memory cells, although their BCR affinity to the wildtype antigen is low. They are ideal for the potential mutations. However, this is less studied in humans, not to mention for the current COVID-19 vaccines.

The inventors have found that variant specific memory B cells (that do not bind for example, the immunizing Spike) have been induced and preserved in human PBMC after two-doses of COVID-19 mRNA vaccination. As provided for in the examples presented herein, the relationship between these variants and wildtype RBD specific B cells were examined and the measure of the percentage of variant specific B cells induced by different types of COVID-19 vaccines was determined. As memory B cells are long lived and can therefore provide long term protection (Palm, A.-K. E. & Henry, C. Remembrance of Things Past: Long-Term B Cell Memory After Infection and Vaccination. Frontiers in Immunology 10, (2019)), understanding the properties of variant specific B cells induced by the wildtype antigen informs vaccine design.

Since the beginning of the SARS-COV-2 pandemic, mAbs and different types of COVID-19 vaccines have been developed to treat virus infected patients and immunize the public respectively. However, these are based on the wildtype spike sequence. As an RNA virus, the SARS-COV-2 is mutating and variants, especially variants of concern and variants of interest, are emerging. All these variants have amino acid substitutions in the spike region. Certain mutations could evade mAbs binding (Greanery, A. J., et al. (2021)) and therefore reduce serum antibody neutralization (Uriu, K. et al. Neutralization of the SARS-COV-2 Mu Variant by Convalescent and Vaccine Serum. New England Journal of Medicine, (2021)). Especially the Omicron variant which has 32 amino acid mutations in the spike region of BA1 version and could escape 80% of serum antibody (Cameroni, E. et al. Broadly neutralizing antibodies overcome SARS-COV-2 Omicron antigenic shift. Nature 602, 664-670, (2022)). Together with the fact that serum antibody is waning after the second dose of mRNA vaccination (Levin, E. G. et al. Waning Immune Humoral Response to BNT162b2 Covid-19 Vaccine over 6 Months. New England Journal of Medicine 385, (2021)), third boost or even fourth boost shot is recommended by FDA for eligible personnel who had been immunized 6 months before (Regev-Yochay, G. et al. Efficacy of a Fourth Dose of Covid-19 mRNA Vaccine against Omicron. New England Journal of Medicine, (2022)). The boost shot still uses the wildtype spike as the immunogen, and therefore could only boost wildtype specific B cell and antibody response. Trials using the Omicron version of mRNA vaccine showed strong neutralization against the Omicron version of virus but low response to other variants including the original virus (Lee, I. J. et al. Omicron-specific mRNA vaccine induced potent neutralizing antibody against Omicron but not other SARS-COV-2 variants. bioRxiv, (2022)). Questions such as how many shots are needed as well as which version of spike should be used for the booster shot(s) need to be urgently addressed.

The effect of successful vaccination is to induce immune memory in the host as the recall immune response is much faster and stronger in case of antigen encounter (Sallusto, F., et al. From Vaccines to Memory and Back. Immunity 33, 451-463, (2010)). This is particularly true for memory B cells as B cells undergo affinity maturation after antigen stimulation, a way to increase the affinity of BCR against the encountered antigen, in the germinal center (Lau, A. W., et al. (2020)). BCR affinity maturation occurs via mutation of the genes coding for the heavy and light chain variable region (V-(D)-J) in the dark zone of the germinal center (Di Noia, J. M. & Neuberger, M. S. Molecular Mechanisms of Antibody Somatic Hypermutation. Annual Review of Biochemistry 76, 1-22, doi: 10.1146/annurev.biochem. 76.061705.090740 (2007); and Mesin, L., et al. Germinal Center B Cell Dynamics. Immunity 45, 471-482, (2016)). During mutation, the germinal center reaction progressively selects for B cells bearing BCRs that react better with the antigen that is present. During the exiting process from germinal center through dark zone, the BCRs of memory B cells might go further rounds of mutations, which generates the antigen diversity on the memory B cells. These memory B cells do not react very well with the immunizing antigen, and yet may react better with related proteins, including variants of the ancestral Spike protein that might otherwise avoid neutralization. These B cells are the subjects of the results disclosed herein.

Such mutation specific memory B cells have been identified in mice after wildtype antigen immunization (Purtha, W. E., et al. (2011)), however little has been studied along these lines in humans. With the mutations in the spike region of SARS-COV-2 variants, the study of mutation specific memory B cells in human PBMC samples after COVID-19 vaccination directly address the concern over the efficacy of current vaccines. As disclosed in the examples presented herein, using double colored mutant RBD tetramers and wildtype a RBD tetramer bearing a third color, mutation specific memory B cells in human PBMCs after the donors have received two-doses of mRNA vaccination have been identified. Further, the relationship between mutation specific B cells and wildtype RBD B cells and whether mutation specific B cells are ubiquitously induced by different types of COVID-19 vaccines is described.

Surprisingly, there are memory B cells specifically recognizing not only one amino acid mutation (spike E484K mutation) or two mutations (spike L452Q and F490S mutations), but also sixty mutations in the case of RBD domain of SARS-COV-1 (Lan, J. et al. Structure of the SARS-COV-2 spike receptor-binding domain bound to the ACE2 receptor. Nature 581, 215-220, (2020)) (full length wild-type Spike sequence is SEQ ID NO:1). As disclosed herein the relationship between mutation specific B cells and wildtype RBD specific B cells is characterized. The inventors believe that mutation specific B cells are generated during the germinal center selection with only a few residues different from the wildtype RBD B cells. To address this, single cell sequencing of the fragment of antigen binding region (Fab) of the B cell receptor from both mutation specific B cells and wildtype RBD specific B cells is performed to identify the sequence relationship between these two B cell populations.

Also disclosed herein is the characterizing of the biological activity of mutation specific B cells and comparing the percentage (or level) of these B cells between different types of COVID-19 vaccines. The inventors believe that mutation specific B cells may not react to vaccine boost using the wildtype spike as the immunogen. Further provided in the examples below, the percentage (or level) of both mutation specific B cells and wildtype RBD B cells in PBMC samples after different doses of mRNA vaccines and after one-dose Ad26.COV2.S adenovirus vaccine is compared.

A successful vaccine could provide long-term protection by inducing immune memories in the host, such as memory T cell and B cells (Sallusto, F., et al. (2010)). In the case of antigen re-exposure, the recall response of memory B cells is much faster and stronger (Goel, R. R., et al. (2022)). The findings disclosed herein help to better understand the perspectives of mutation specific B cells after wildtype antigen immunization and further benefit vaccine design and vaccination scheduling strategy.

In one aspect, comparison of the antibody sequences of B cells that bind Spike variants, but not, detectably, the wild-type Spike, after vaccination is contemplated. It is believed that the variant specific cells will have antibody sequences that are clearly derived from those that bind the wild-type Spike, since it is believed that the variant specific cells are derived by somatic mutation in germinal centers from cells stimulated by the wild-type antigen. As memory B cells are key indicators of long-term protection, assessing both wild-type and mutation specific memory B cells are critical factors evaluating the efficacy of vaccination, especially against the emerging virus variants. Disclosed herein are variant specific memory B cells in human PBMCs that appear after two-doses of a COVID-19 mRNA vaccination. Together with the stability of memory B cells, the efficacy of vaccines in terms of inducing stable levels of long-lived memory B cells level with the ability of reacting with Spike variants is assessed. Such cells are particularly important since, with appearance of the virus, they can replenish the rapidly waning levels of serum antibody in response to the virus.

In one aspect, this disclosure provides an isolated anti-SARS-COV-2 antibody or antigen-binding fragment thereof that binds specifically to a SARS-COV-2 antigen. In some embodiments, the SARS-COV-2 antigen comprises a portion of a Spike (S) polypeptide, such as an S polypeptide of a human or an animal SARS-COV-2. In some embodiments, the SARS-CoV-2 antigen comprises the receptor binding domain (RBD) of the S polypeptide. In some embodiments, the RBD comprises amino acids 319-541 (of SEQ ID NO:1) of the S polypeptide.

The spike protein is important because it is present on the outside of intact SARS-CoV-2. Thus, it presents a target that can be used to inhibit or eliminate an intact virus before the virus has an opportunity to infect a cell. A representative amino acid sequence is provided below: (SEQ ID NO:1)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The disclosure further provides a method of determining the efficacy of a COVID-19 vaccine to a SARS-COV-2 variant on the perspective of memory B cells, comprising the steps of: (i) contacting a sample comprising peripheral blood mononuclear cells (PBMC) with a wild-type RDB tetramer; (ii) further contacting the sample with a variant RDB tetramer; (iii) determining binding percentage and/or level of the RBD tetramers to B cells in the samples, wherein a reduction in the binding percentage and/or level to the B cells of the variant RBD tetramer as compared to the binding of the B cells in the wild-type tetramer indicates less efficacy of the COVID-19 vaccine to the SARS-COV-2 variant.

In some embodiments, the SARS-COV-2 antigen comprises an S polypeptide, such as a S polypeptide of a human or an animal SARS-COV-2. In some embodiments, the SARS-CoV-2 antigen comprises the receptor-binding domain (RBD) of the S polypeptide. In some embodiments, the RBD comprises amino acids 319-541 of the S polypeptide.

In some embodiments, the antibody or antigen binding fragment, or tetramer is conjugated to a label. In some embodiments, the step of detecting comprises contacting a secondary antibody with the antibody or antigen-binding fragment and wherein the secondary antibody comprises a label. In some embodiments, the label includes a fluorescent label, a chemiluminescent label, a radiolabel, and/or an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and/or an affinity purification column.

Samples that can be used in the assays according to the present invention include any tissue or fluid sample obtainable from a patient. Samples may be obtained from a healthy patient (e.g., a patient not afflicted with a virus) to initially establish a baseline, or standard, level. Further, a baseline or standard sample can be obtained from a patient prior to administration of a viral vaccine. This baseline level can then be compared against the levels of samples obtained from individuals suspected of having a viral-associated condition, or symptoms associated with such condition, or against samples from patients that have received a viral vaccine. The methods disclosed herein can measure serum antibody and/or memory B cells against a viral protein, such as against SARS-COV-2 spike, specifically RBD. The methods disclosed herein can measure the antibody or B cell response after vaccination or virus infection. A preferred sample is a PBMC sample.

The antibodies and/or antigen binding fragments and/or tetramers of this invention represent an excellent way for the development of antiviral therapies either alone or in antibody cocktails with additional viral antibodies (including but not limited to anti-SARS-COV-2 virus antibodies) for the treatment of viral infections in humans (including but not limited to SARS-CoV-2 infections).

In another aspect, the present invention provides a pharmaceutical composition comprising the antibodies and/or the antigen binding fragments of the present invention described herein formulated together with a pharmaceutically acceptable carrier. The composition may optionally contain one or more additional pharmaceutically active ingredients, such as another antibody or a therapeutic agent.

The pharmaceutical compositions of the invention also can be administered in a combination therapy with, for example, another immune-stimulatory agent, an antiviral agent, or a vaccine, etc.

Also within the scope of this disclosure is use of the pharmaceutical composition in the preparation of a medicament for the diagnosis, prophylaxis, treatment, or combination thereof of a condition resulting from viruses including but not limited to SARS-COV-2 and flu.

The term “antigen-binding fragment or portion” of an antibody (or simply “antibody fragment or portion”), as used herein, refers to one or more fragments of an antibody that retain the ability to specifically bind to an antigen (e.g., a Spike or S protein of SARS-COV-2 virus or BCR of B cells).

The term “detectable label” as used herein refers to a molecule capable of detection, including, but not limited to, radioactive isotopes, fluorescers, chemiluminescers, chromophores, enzymes, enzyme substrates, enzyme cofac-tors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

An “individual” is a vertebrate, such as a mammal, including without limitation a human. Mammals include, but are not limited to, farm animals, sport animals, pets, primates, mice and rats. The term “individual” can be used interchangeably with the term “animal”, “subject” or “patient”.

As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but has a different structure or origin with respect to the reference compound.

The terms “substituted”, “substituted derivative” and “derivative”, when used to describe a compound, means that at least one hydrogen bound to the unsubstituted compound is replaced with a different atom or a chemical moiety.

Although a derivative has a similar physical structure to the parent compound, the derivative may have different chemical and/or biological properties than the parent compound. Such properties can include, but are not limited to, increased or decreased activity of the parent compound, new activity as compared to the parent compound, enhanced or decreased bioavailability, enhanced or decreased efficacy, enhanced or decreased stability in vitro and/or in vivo, and/or enhanced or decreased absorption properties.

In general, the term “biologically active” indicates that a compound (including a protein or peptide) has at least one detectable activity that has an effect on the metabolic, physiological, chemical, or other processes of a cell, a tissue, or an organism, as measured or observed in vivo (i.e., in a natural physiological environment) or in vitro (i.e., under laboratory conditions).

According to the present invention, the term “modulate” can be used interchangeably with “regulate” and refers generally to upregulation or downregulation of a particular activity. As used herein, the term “upregulate” can be used generally to describe any of: elicitation, initiation, increasing, augmenting, boosting, improving, enhancing, amplifying, promoting, or providing, with respect to a particular activity. Similarly, the term “downregulate” can be used generally to describe any of: decreasing, reducing, inhibiting, ameliorating, diminishing, lessening, blocking, or preventing, with respect to a particular activity.

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

According to the present invention, the phrase “selectively binds to” refers to the ability of an antibody, antigen-binding fragment, tetramer or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art including enzyme immunoassays (e.g., ELISA, immunoblot assays, etc.).

General reference to a protein or polypeptide used in the present invention includes full-length proteins, or any fragment, domain (structural, functional, or immunogenic), conformational epitope, or a homologue or variant of a given protein. A fusion protein may also be generally referred to as a protein or polypeptide. An isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein of the present invention is produced recombinantly. According to the present invention, the terms “modification” and “mutation” can be used interchangeably, particularly with regard to the modifications/mutations to the amino acid sequence of proteins or portions thereof (or nucleic acid sequences) described herein.

As used herein, the term “homologue” or “variant” is used to refer to a protein or peptide (including an antibody) which differs from a reference protein or peptide (i.e., the “prototype” or “wild-type” protein) by minor modifications to the reference protein or peptide or antibody, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue or variant can have enhanced, decreased, or substantially similar properties as compared to the reference protein or peptide. A homologue or variant can include an agonist of a protein or an antagonist of a protein. Homologues or variants can be produced using techniques known in the art for the production of proteins including, but not limited to, direct modifications to the isolated reference protein, direct protein synthesis, or modifications to the nucleic acid sequence encoding the protein using, for example, classic or recombinant DNA techniques to effect random or targeted mutagenesis, resulting in the encoding of a protein variant. In addition, naturally occurring variants of a reference protein may exist (e.g., isoforms, allelic variants, or other natural variants that may occur from individual to individual) and may be isolated, produced and/or utilized in the invention.

A homologue or variant of a given protein may comprise, consist essentially of, or consist of, an amino acid sequence that is at least about 45%, or at least about 50%, or at least about 55%, or at least about 60%, or at least about 65%, or at least about 70%, or at least about 75%, or at least about 80%, or at least about 85%, or at least about 86% identical, or at least about 87% identical, or at least about 88% identical, or at least about 89% identical, or at least about 90%, or at least about 91% identical, or at least about 92% identical, or at least about 93% identical, or at least about 94% identical, or at least about 95% identical, or at least about 96% identical, or at least about 97% identical, or at least about 98% identical, or at least about 99% identical (or any percent identity between 45% and 99%, in whole integer increments), to the amino acid sequence of the reference protein (e.g., an amino acid sequence specified herein, or the amino acid sequence of a specified protein). In one embodiment, the homologue or variant comprises, consists essentially of, or consists of, an amino acid sequence that is less than 100% identical, less than about 99% identical, less than about 98% identical, less than about 97% identical, less than about 96% identical, less than about 95% identical, and so on, in increments of 1%, to less than about 70% identical to the amino acid sequence of the reference protein.

As used herein, unless otherwise specified, reference to a percent (%) identity refers to an evaluation of homology which is performed using: (1) a Basic Local Alignment Search Tool (BLAST) basic homology search using blastp for amino acid searches and blastn for nucleic acid searches with standard default parameters, wherein the query sequence is filtered for low complexity regions by default (such as described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST alignment of two sequences (e.g., using the parameters described below); (3) and/or PSI-BLAST with the standard default parameters (Position-Specific Iterated BLAST. It is noted that due to some differences in the standard parameters between Basic BLAST and BLAST for two sequences, two specific sequences might be recognized as having significant homology using the BLAST program, whereas a search performed in Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST as described in Tatusova and Madden, (1999), “Blast 2 sequences—a new tool for comparing protein and nucleotide sequences”, FEMS Microbiol Lett. 174:247-250, incorporated herein by reference in its entirety. Such a sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST sequence alignment for two sequences is performed using the standard default parameters as follows.

$$\begin{matrix}For blastn , using 0 BLOSUM 62 matrix : \\ Reward for match = 1 \\ Penalty for mismatch = - 2 \\ Open gap ( 5 ) and extension gap ( 2 ) penalties \\ gap x\_dropoff ( 50 ) expect ( 10 ) word size ( 11 ) filter ( on )\end{matrix}$$

$$\begin{matrix}For blastp , using 0 BLOSUM 62 matrix : \\ Open gap ( 11 ) and extension gap ( 1 ) penalties \\ gap x\_dropoff ( 50 ) expect ( 10 ) word size ( 3 ) filter ( on ) .\end{matrix}$$

An isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome or a segment of the genome containing more than one gene, in which the nucleic acid molecule is found in nature. An isolated nucleic acid molecule can include a complete gene. An isolated nucleic acid molecule that includes a gene is not a fragment of a chromosome that includes such gene, but rather includes the coding region and regulatory regions associated with the gene, but no additional genes that are naturally found on the same chromosome. An isolated nucleic acid molecule may also include portions of a gene. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

A recombinant nucleic acid molecule is a molecule that can include at least one of any nucleic acid sequence encoding any one or more proteins described herein operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

A recombinant nucleic acid molecule includes a recombinant vector, which is any nucleic acid sequence, typically a heterologous sequence, which is operatively linked to the isolated nucleic acid molecule encoding a fusion protein of the present invention, which is capable of enabling recombinant production of the fusion protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a plasmid useful for transfecting yeast. Recombinant vectors can be used in the cloning, sequencing, and/or otherwise manipulating of nucleic acid molecules, and can be used in delivery of such molecules (e.g., as in a DNA composition or a viral vector-based composition). Recombinant vectors are preferably used in the expression of nucleic acid molecules, and can also be referred to as expression vectors. Preferred recombinant vectors are capable of being expressed in a transfected host cell, such as a yeast.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more expression control sequences. The phrase “operatively linked” refers to linking a nucleic acid molecule to an expression control sequence in a manner such that the molecule is expressed when transfected (i.e., transformed, transduced or transfected) into a host cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast. In microbial systems, the term “transformation” is used to describe an inherited change due to the acquisition of exogenous nucleic acids by the microorganism and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

The following experimental results are provided for purposes of illustration and are not intended to limit the scope of the invention.

### EXAMPLES

### Example 1

Methods for the examples described below.

### SARS-COV-2 Spike Receptor Binding Domain (RBD) Gene Cloning and Transfection:

DNA sequence encoding the SARS-COV-2 spike leader sequence (spike amino acid 1 to 14) fused to wildtype spike RBD region (spike amino acids 319 to 541) followed by 8 histidine tag and Avi tag was synthesized (see SEQ ID NO:2 below with the leader sequence underlined). The gene was amplified by PCR, digested with NheI/XhoI enzymes and ligated to pcDNA3.1 vector digested with the same NheI/XhoI enzymes. The inserted DNA sequence was verified by DNA sequencing.

SEQ ID NO: 2
MFVFLVLLPLVSSQVQPTESIVRFPNITNLCPFGEVENATRFASVYAWNR
KRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGY
QPYRVVVLSFELLHAPATVCGPKKSTNLVKNKCVNFHHHHHHHHGLNDIF
EAQKIEWHE

Variant RBD expression vector was generated with quick change mutagenesis of pcDNA3.1 wildtype RBD vector and also verified by DNA sequencing.

Vectors containing wildtype or mutant RBD were transient transfected into HEK 293F cells with polyethylenimine (PEI) for RBD protein expression.

### Spike RBD Protein Purification, Biotinylation and Tetramer Conjugation:

The RBD protein was purified using a nickel column from supernatant five days after transfection. RBD was further purified by passing through a size column to collect the monomer peak.

The RBD protein has the Avi tag, which is biotinylated at lysine site by BirA enzyme.

The biotinylated RBD is conjugated to fluorescent dye labeled streptavidin. The molar ration between biotinylated RBD and streptavidin is at 6:1 to ensure saturation of streptavidin as one streptavidin can bind four biotins. The excess unbound RBD was discarded by flowing through an AMICON® concentration column with 100 kD cutoff by centrifugation.

The protein concentration of RBD-tetramer is measured by BIORAD® protein assay dye. The tetramer is stored in PBS buffer with 15% of glycerol at −80 degree freezer.

### Human B Cell Staining in Peripheral Blood Mononuclear Cells (PBMC):

The PBMC samples are from a biobank at National Jewish Health, which processes donated blood samples and stores PBMC samples at −80 freezers for long term storage. The preparation of PBMC samples from whole blood is by using the Ficoll density gradient centrifugation. The PBMCs will stay in the middle of media after centrifugation. PBMC samples from donations before the COVID-19 outbreak or before the COVID-19 vaccination as the negative control are used. Ideally, PBMC samples from the same donors before and after COVID-19 vaccination are used to analyze vaccine induced B cell immunity.

PBMC samples are thawed at 37° C. water bath and washed once with PBS buffer. The cells are then spun down at 1500 rpm for 5 minutes and the supernatant is discarded. The cell pellet is re-suspended in BSS wash buffer, counted by cell counter for cell number and distributed into multiple wells of 96-well plate for staining.

2 μg/ml double colored wildtype RBD (wildtype RBD conjugated to streptavidin labeled with BV421 or with Phycoerythrin (PE)), 2 μg/ml variant RBD tetramer with a third color (variant RBD conjugated to streptavidin labeled with BV711), 2 g/ml OVA-FITC and human Fc receptor block antibody (at 1:200 dilution) cocktail is added to the PBMC samples and incubated on ice for 30 minutes. Similarly, double colored variant RBD (variant RBD conjugated to streptavidin labeled with BV711 or PE) and wildtype RBD tetramer with a third color (wildtype RBD conjugated to streptavidin labeled with BV421), OVA-FITC and human Fc receptor block antibody cocktail is also added to a different well for staining.

After cocktail staining, 1 g/ml CD19 antibody labeled with APCcy7, IgD antibody labeled with BV510, dump (CD4, CD8, CD14, CD16) antibody labeled with PerCP are then added for staining on ice for another 30 minutes. Cells are spun down and the supernatant is discarded. The cells are washed once with PBS buffer and stained with Ghost UV450 dye and fixed with 1% paraformaldehyde at 4° C. overnight.

### Flow Cytometry and Data Analysis:

The cell staining is analyzed by LSRFortessa cell analyzer (BD BIOSCIENCE®). This machine has 5 lasers (blue, red, violet, UB and yellow-green) and can read up to 18 different colors. Antibodies usually have affinity against monomeric antigen at nano-molar range. A cell will bind antibody as long as it expresses the corresponding antigen. B cells have surface immunoglobulin expression as B cell receptors (BCR). BCR will bind RBD-streptavidin tetramers if the affinity of BCR against RBD reaches a certain range. Therefore, the amount of antibody or RBD-tetramer binding to a certain cell correlates with the amount of antigen expression and the affinity of antibody. The color from the fluorescent dye, either from tetramer or antibody staining, on a single cell is excited by lasers and recorded by the cell analyzer.

The data analysis of cell staining is done by FLOWJO® software. Single cells are first chosen based on the forward scatter (FSC) and side scatter (SSC) signal of cells recorded by the optical detectors of LSRFortessa. Live cells are then gated on those with Ghost UV450 negative staining. Cells other than B cells are excluded by positive of CD4/8/14/16 PerCP staining. B cells are then selected by CD19 positive staining. The non-specific binding B cells are then excluded by the OVA-FITC positive staining.

From the remaining B cells, the wildtype RBD tetramer signal is analyzed. The double wildtype RBD tetramer positive gate is drawn in the case of double wildtype RBD tetramer and variant RBD tetramer with a third color. The setting of the gate is also based on PBMC samples before the COVID-19 vaccination, as these negative control samples should have no or very low double positive RBD tetramer staining. Once the gate is set, it applies to all the staining samples.

The variant RBD tetramer binding signal from the double colored RBD tetramer binding B cells is then analyzed.

The double variant RBD double positive gate can be drawn in the same way in the case of double variant RBD tetramers and wildtype RBD tetramer with a third color staining. The wildtype RBD tetramer signal is then analyzed from the double variant RBD binding B cell gate.

Double colored wildtype RBD tetramers and variant RBD tetramer with a third color staining can identify wildtype RBD specific B cells and variant RBD cross-reactive B cells. Double colored variant RBD tetramers and wildtype RBD tetramer with a third color can identify mutation specific B cells and cross-reactive B cells. The staining can also be done by using the double-colored wildtype RBD streptavidin tetramers (such as BV421 and PE) and double colored variant RBD streptavidin tetramers with third and fourth color (such as BV711 and APC) in the same staining condition as long as all four antigen streptavidin tetramers are available.

### Example 2

The following example describes the discovery of the relationship between mutant specific B cells and wildtype specific RBD B cells.

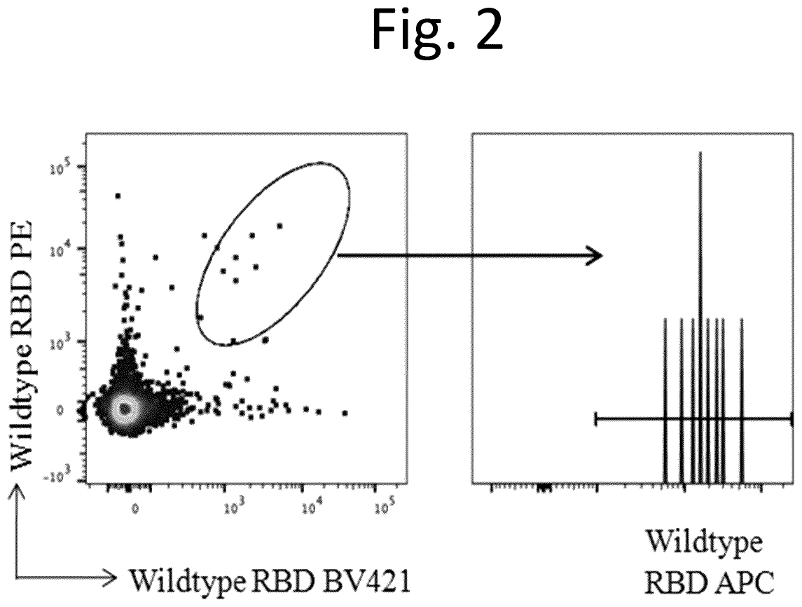

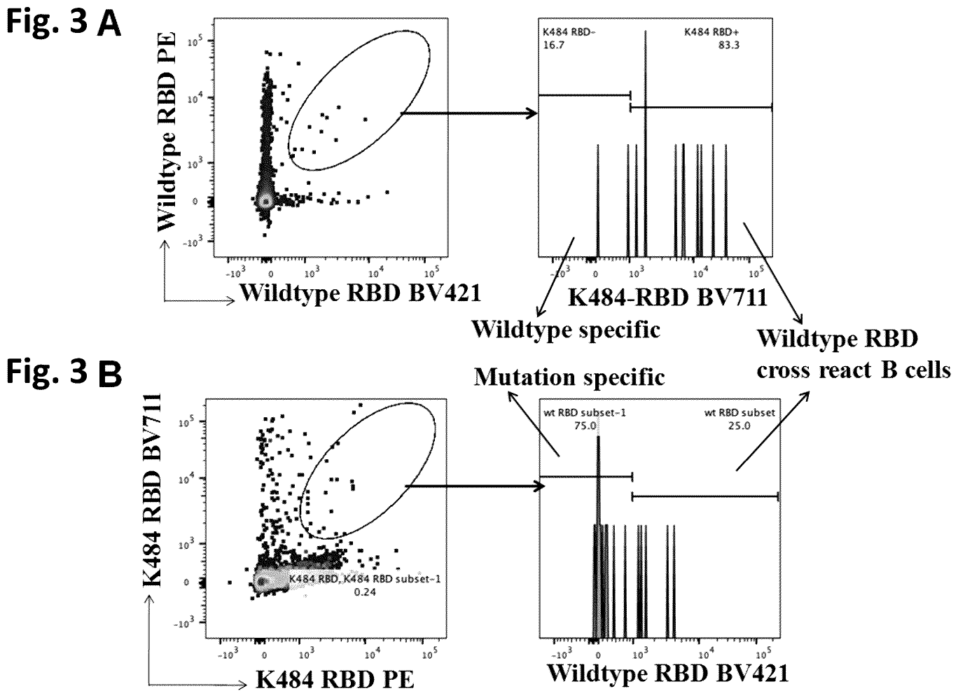

Amino acid mutation(s) in the variant escapes antibody binding by breaking the micro-environment interaction between the antibody and wildtype antigen. The extent of escape depends not only on how much the original amino acid contributes to the interaction between antibody and antigen, but also on how much the mutation breaks the original interaction (Starr, T. N., et al. Complete map of SARS-COV-2 RBD mutations that escape the monoclonal antibody LY-CoV555 and its cocktail with LY-CoV016. Cell Reports Medicine 2, 100255, (2021)). For example, switching side chain polarity from a negative to a positive charge might be particularly effective since salt bridge interactions between antigen and antibody contribute most to high affinity interactions (Erijman, A., et al. How Structure Defines Affinity in Protein-Protein Interactions. PLOS ONE 9, (2014)). Because RBD monomers often dissociate from BCRs quickly, monomeric RBD cannot be used reliably to identify RBD specific B cells. Thus instead, RBD tetramers were used along the same lines as these are used to identify MHC/peptide specific T cells. Additionally double colored RBD tetramers were used bearing the same RBD to identify RBD specific B cells in PBMCs and eliminate non-specific binding (Wang, Z. et al. Naturally enhanced neutralizing breadth against SARS-COV-2 one year after infection. Nature 595, 426-431, (2021)). This staining method proves to have high antigen specificity (FIG. 2). As described herein, three tetramers each bearing a different color are used. Two of these bore the same wildtype RBD and the third, a mutant RBD. For example, equal amounts of wildtype RBD tetramer in two different colors and mutant RBD (K484-RBD) tetramer with a third color was used to investigate the effect of E484K mutation on BCR binding on the B cell level. A single E484K amino acid mutation abolished around 16% of wildtype specific B cell binding, thus the cells that bind the wild type RBD but not the mutant RBD can be defined as wildtype specific B cells (FIG. 3A).

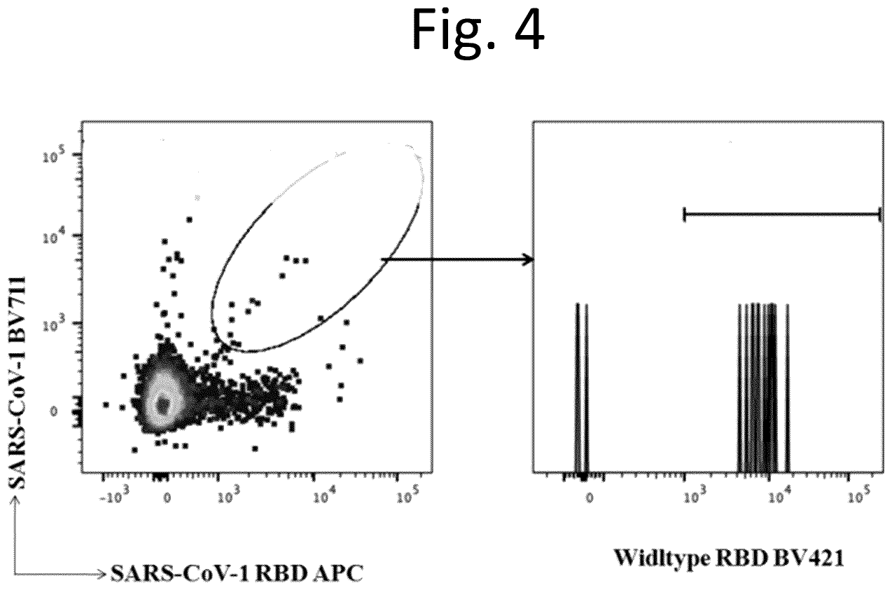

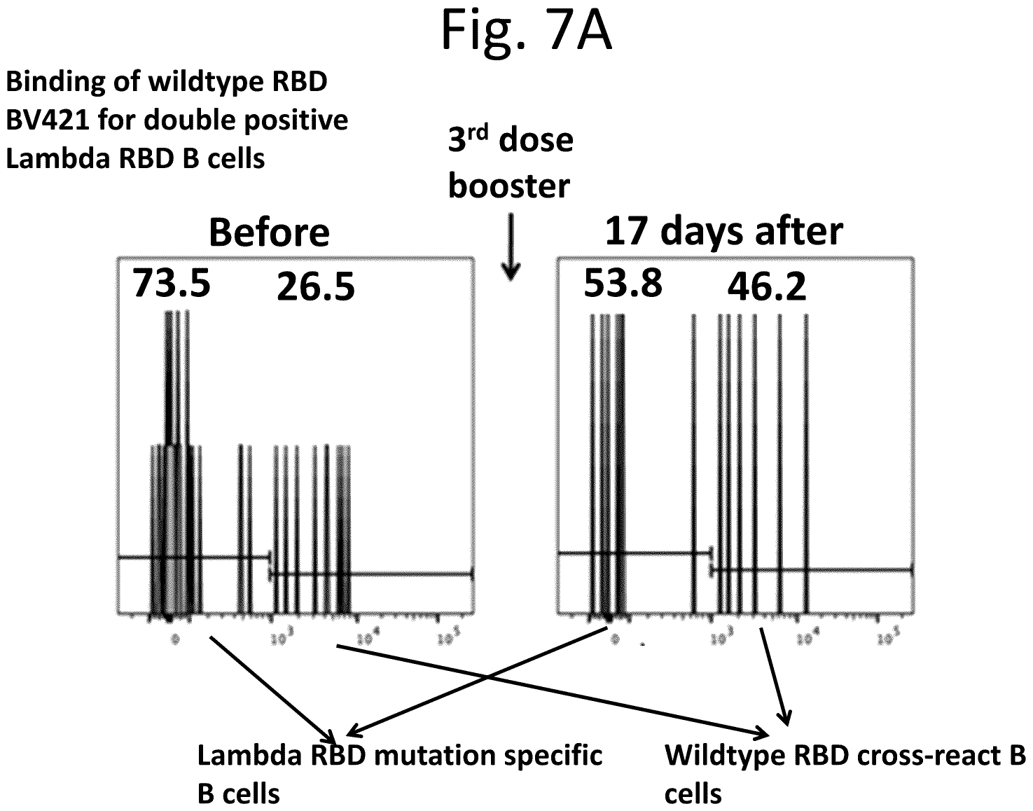

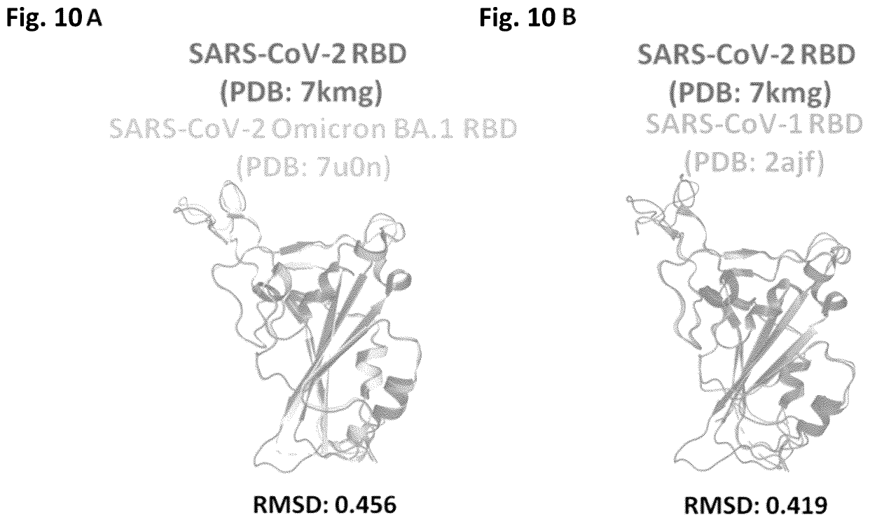

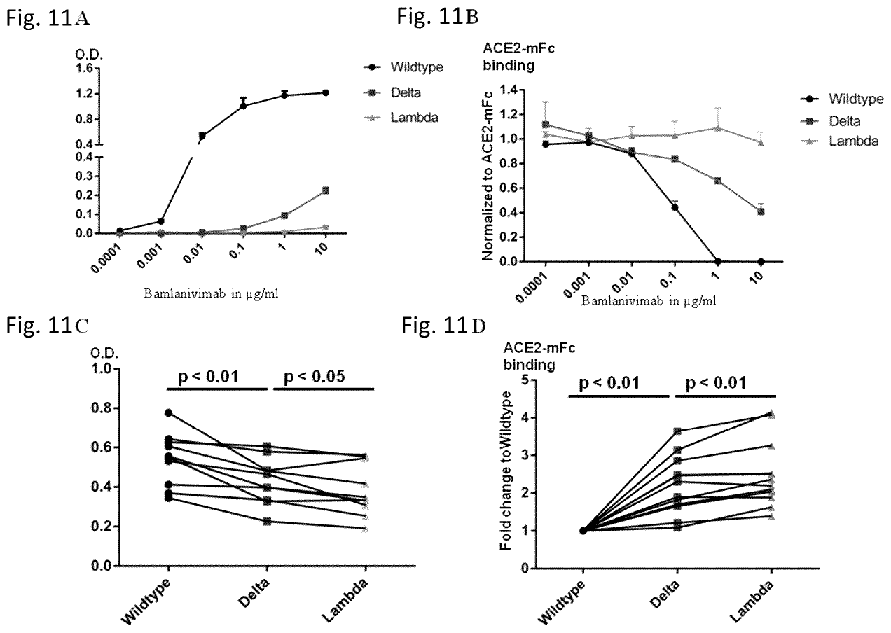

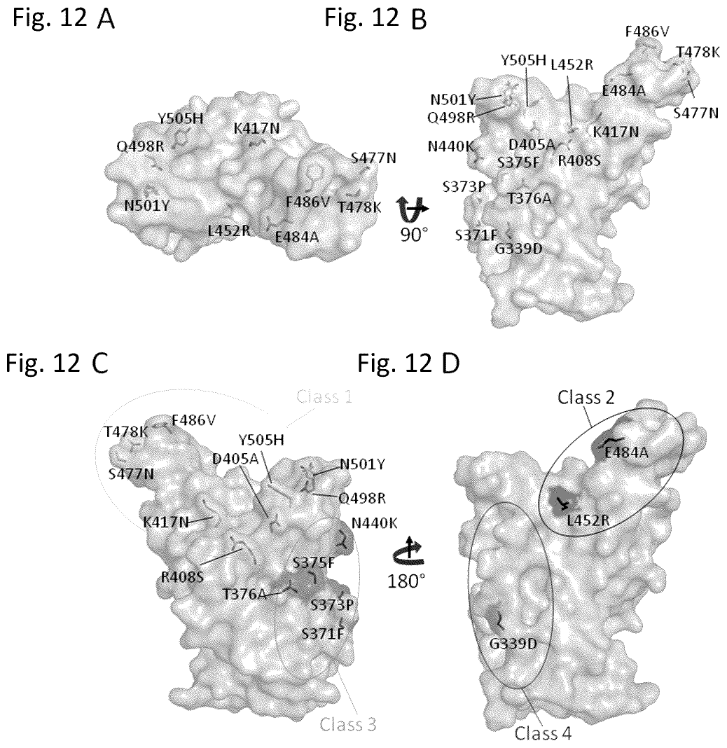

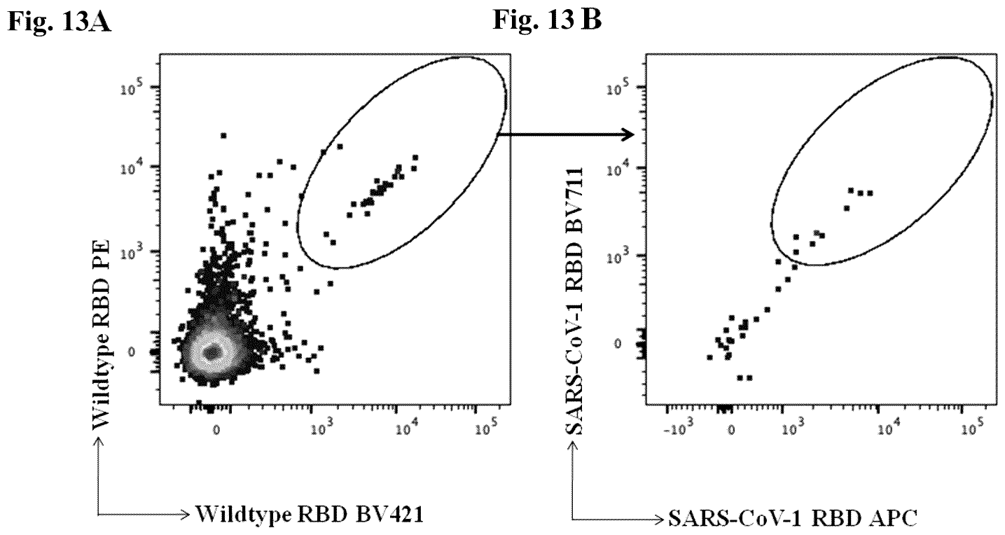

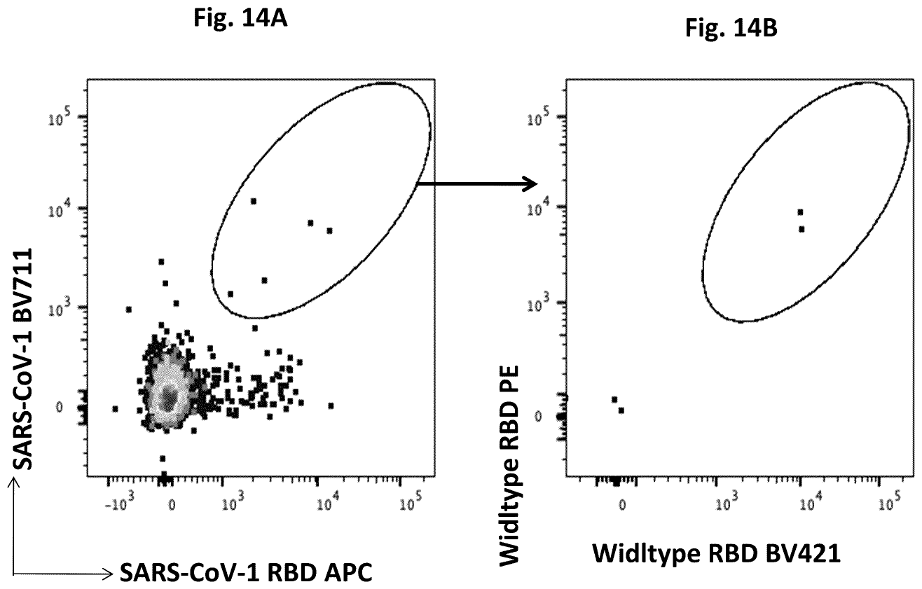

It has been shown that in mice some memory B cells can recognize mutations, but not the original version of a viral protein for immunization in mice (Purtha, W. E. et al. (2011)). To find out whether this phenomenon occurs also in humans responding to two-doses of COVID-19 mRNA vaccination, a double-colored mutant RBD (K484-RBD) tetramer and wildtype RBD tetramer with a third color was used to differentiate mutant RBD binding B cells into those that reacted only with a mutant Spike from those that were wildtype RBD cross-reactive B cells (FIG. 3B). It was found that mutation specific B cells were generated not only for the E484K mutation, but also for the Lambda variant RBD with two mutations (L452Q and F490S mutations shown in FIG. 7), and even for the SARS-COV-1 RBD with 59 amino acid mutations and one amino acid deletion compared to the SARS-COV-2 wildtype RBD (FIG. 4). Clearly, two-dose COVID-19 mRNA vaccine using the wildtype spike as the immunogen induced human memory B cells against different RBD mutations, even against SARS-COV-1 RBD with similar protein backbone structure as SARS-COV-2 spike (FIG. 10).

To determine what the sequence similarity in the Fab region between mutation specific B cells and wildtype specific B cells is and to determine if they share most of their sequence except the key amino acids contacting with the wildtype and mutant amino acid, single cell B cell receptor (BCR) sequencing is used for the K484-RBD binding B cells without binding wildtype RBD (mutation specific) and wildtype RBD binding B cells without K484-RBD binding (wildtype specific). This is addressed by the single B cell immune profiling on the 10* platform using FACS sorted B cells after staining as described above based on their ability to react with mutant only versus wildtype only versus cross reactive from the same donor.

### Example 3

The following example describes studies on the longevity of mutation specific memory B cells compared with wildtype RDB memory B cells.

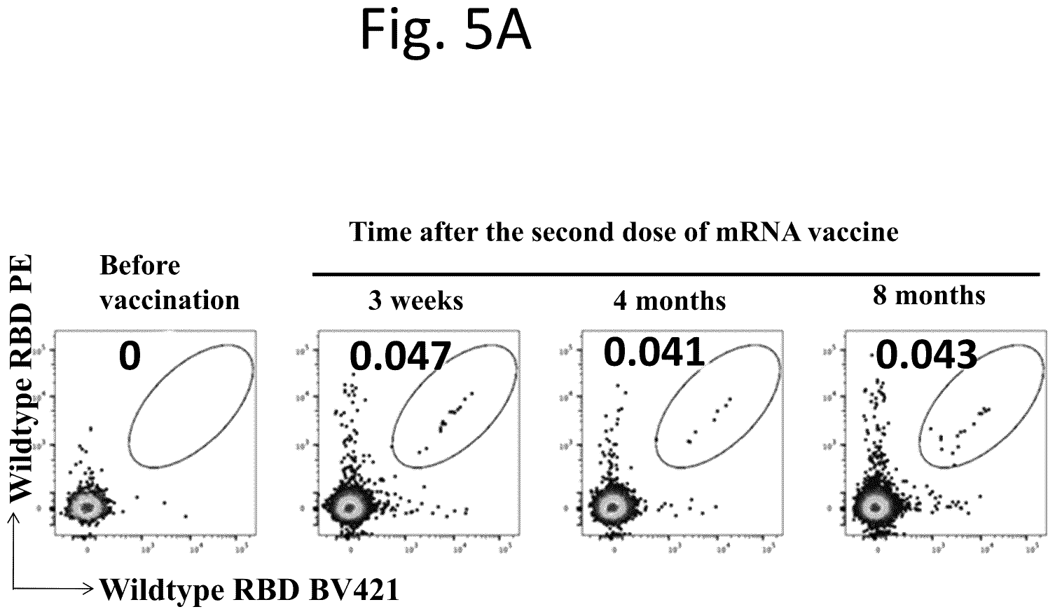

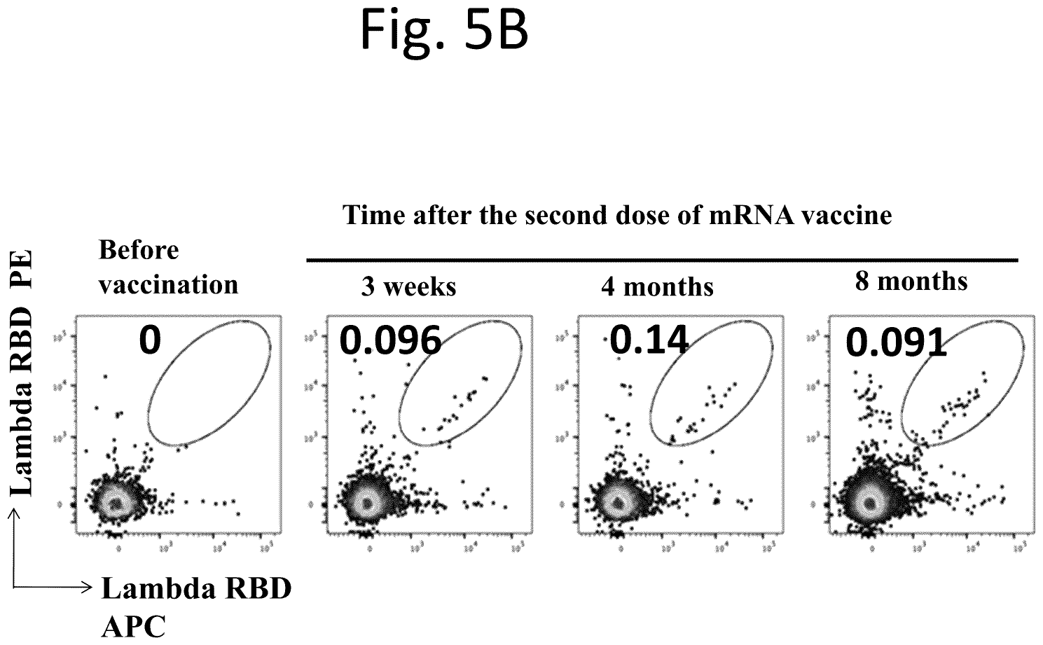

With mutation specific memory B cells identified in human PBMCs after COVID-19 mRNA vaccination, another question to address is whether these memory B cells have the same longevity as the wildtype RBD binding memory B cells. As there is no clear B cell surface marker on human memory B cells for longevity (Palm, A.-K. E., et al. (2019)), the foremost marker for antigen specific memory B cells is antigen binding. The percentage of wildtype RBD binding B cells and Lambda RBD binding B cells from same donor at different time points after a second dose of mRNA vaccine was determined, both of which are relatively stable (FIG. 5).

As memory B cells are long-lived due to expressing transcriptional factors such as Bcl-2 (Nuñez, G., et al. (1991)), assessing the amount of transcription factor is an ideal way to compare the longevity of mutation specific B cells and wildtype antigen memory B cells. Using the single B cell immune profiling on the 10* platform will get the mRNA transcription data on the single cell level. The mRNA expression level of long-lived transcription factors between the mutation specific memory B cells and wildtype specific memory B cells is compared. Thus, the relative longevity of mutation specific B cells compared with wildtype RBD memory B cells is determined.

Mutation specific B cells and wildtype specific B cells are single cell sequenced on the 10* platform to find out the relationship between mutation specific B cells and wildtype RBD binding B cells in order to identify Fab usage and longevity based on the transcription factor expression.

To validate the conclusion that some memory B cells bind a variant but react poorly or not at all with the RBD expressed by the vaccine, the light and heavy chain sequences from B cells of interest are cloned and expressed in HEK293 cells. Secreted antibody is purified and its affinity for the variant and ancestral version of the RBD is measured (see Liu, H. et al. The basis of a more contagious 501Y.VI variant of SARS-COV-2. Cell Research 31, 720-722, (2021)). Similar preparations are made for antibodies that react well with the vaccine RBD and have sequences related to the variant specific antibodies. These results demonstrate that the memory B cells that react better with the variant than the vaccine antigen might be related to the latter.

### Example 4

This example shows the characterization of the biological activity of mutation specific B cells and compares the percentage of these B cells between different types of COVID-19 vaccines.

Several types of vaccines have been developed to immunize the public. In the U.S., full-length spike based adenovirus vaccine (JANSSEN® Ad26.COV2.S) (Sadoff, J. et al. Safety and Efficacy of Single-Dose Ad26.COV2.S Vaccine against Covid-19. New England Journal of Medicine 384, 2187-2201, doi: 10.1056/NEJMoa2101544 (2021)) or mRNA vaccines (PFIZER® BNT162b2 and MODERA® mRNA-1273) (Polack, F. P. et al. (2020); Baden, L. R. et al. (2020)) have been approved. Additionally, some donors used herein have been immunized with the ASTRAZENICA® vaccine (AZD1222) (Falsey, A. R., et al. (2021)) or the NOVOVAX® vaccine (Heath, P. T. et al. Safety and Efficacy of NVX-CoV2373 Covid-19 Vaccine. New England Journal of Medicine 385, 1172-1183, (2021)). As variant specific memory B cells have been identified after two-doses of mRNA vaccines, the biological activity of mutation specific B cells is further studied to extend such research into other types of COVID-19 vaccines. Since the purpose of vaccination is to build up long-term memory, the memory B cell response (Sallusto, F., et al. (2010)) is assessed, both for the mutation specific B cells and wildtype memory B cells in different types of vaccines.

The timing of mutation specific B cells generation and whether boost with wildtype immunogen could increase the percentage of this population is determined. As the memory B cells are generated after the initial antigen encounter (Victora, G. D. & Nussenzweig, M. C. Germinal Centers. Annual Review of Immunology 40, 413-442, (2022)), chances are that mutation specific memory B cells appear during the time when the wildtype antigen specific memory B cells exit the germinal center through the dark zone by additional rounds of mutations on their BCR. Both wildtype and mutation specific memory B cells may become long lived and circulate in the peripheral lymphoid organs to survey the antigens (Lau, A. W., et al. (2020)). These cells would proliferate into antibody secreting plasma B cells in case of antigen re-exposure either in the form of repeat vaccination (Muecksch, F. et al. Increased memory B cell potency and breadth after a SARS-COV-2 mRNA boost. Nature, (2022)) or breakthrough infection (Terreri, S. et al. Persistent B cell memory after SARS-COV-2 vaccination is functional during breakthrough infections. Cell Host & Microbe 30, 400-408.e404, (2022)), emphasizing the importance of inducing antigen specific memory B cells by vaccines.

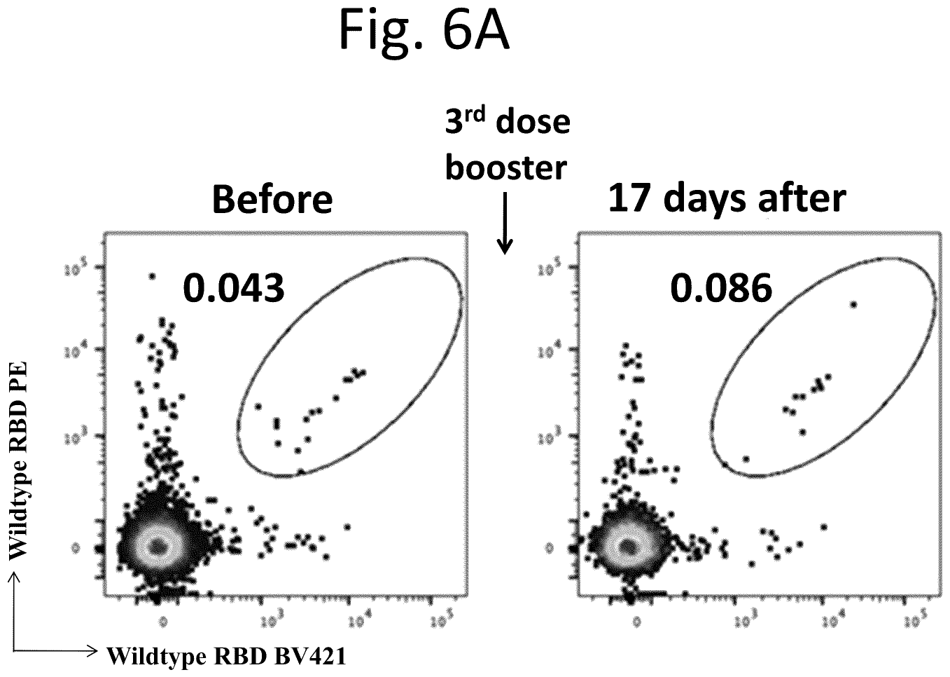

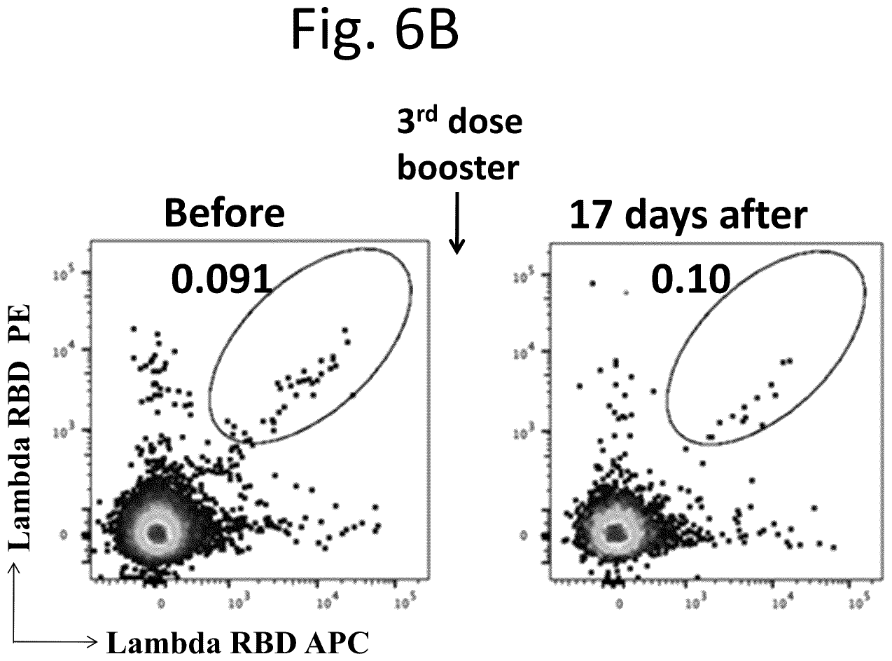

The percentage of wildtype RBD B cells have been reported to increase after boost due to the recall response of memory B cells (Muecksch, F. et al. (2022)). But little is known about the response of mutation specific memory B cells to wildtype immunogen. Preliminary data suggests that the percentage of wildtype RBD binding B cells doubles after boost (FIG. 6A), but the Lambda (variant) RBD binding B cells increased only 10% after boost in the same donor (FIG. 6B). With the double Lambda RBD tetramer and wildtype RBD tetramer staining, the Lambda RBD binding B cells are further divided into Lambda mutation specific B cells and wildtype RBD cross-reactive B cells (FIG. 7A). Importantly, the increase of Lambda RBD binding B cells results from the increase of wildtype RBD cross-react B cells, which is also doubled in percentage in well agreement with that of wildtype RBD B cells, while the percentage of Lambda mutation specific binding B cells decreased by 18% due to the expansion of wildtype RBD B cells (FIG. 7B). This is fully expected as the BCR on the memory B cells possesses the antigen specificity and should only be stimulated by its corresponding paratope on the antigen. Therefore, a double-colored mutant RBD tetramer together with wildtype RBD tetramer with a third color is used to stain PBMC samples collected from same donors after first dose, second dose and third boost of mRNA vaccination to calculate both the mutation specific and wildtype RBD memory B cells.

Mutation specific and wildtype RBD memory B cells after mRNA or adenovirus vaccines are compared. As mutation specific memory B cells after two-dose mRNA vaccination have been identified, research to other types of COVID-19 vaccines is extended to investigate whether there is any difference in inducing mutation specific memory B cells by different types of vaccines.

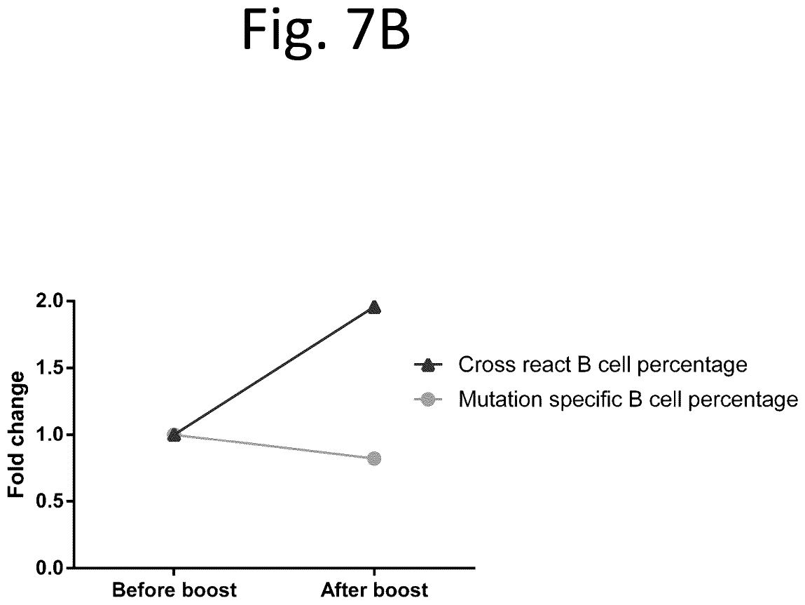

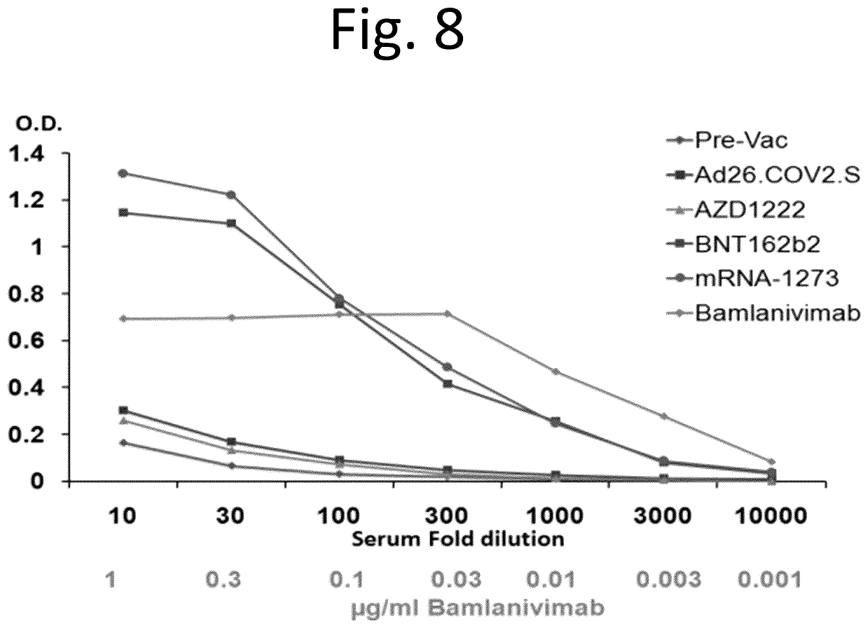

The neutralization titer of serum antibody after mRNA vaccination is much higher than after adenovirus or inactivated virus vaccine (Dashdorj, N. J. et al. Direct comparison of antibody responses to four SARS-COV-2 vaccines in Mongolia. Cell Host & Microbe 29, 1738-1743, (2021)). This correlates with the serum antibody affinity and amount after different types of vaccines (Khoury, D. S. et al. Neutralizing antibody levels are highly predictive of immune protection from symptomatic SARS-COV-2 infection. Nature Medicine 27, 1205-1211, (2021)). The serum antibody level can be accurately measured by an ELISA assay with Bamlanivimab as standard (Liu, H. et al. The Lambda variant of SARS-COV-2 has a better chance than the Delta variant to escape vaccines. bioRxiv, 2021.2008.2025.457692 (2021)). (FIG. 8). The serum antibody after mRNA vaccination is much higher after mRNA vaccination than after AZD1222 and Ad26.COV2.S (FIG. 8).

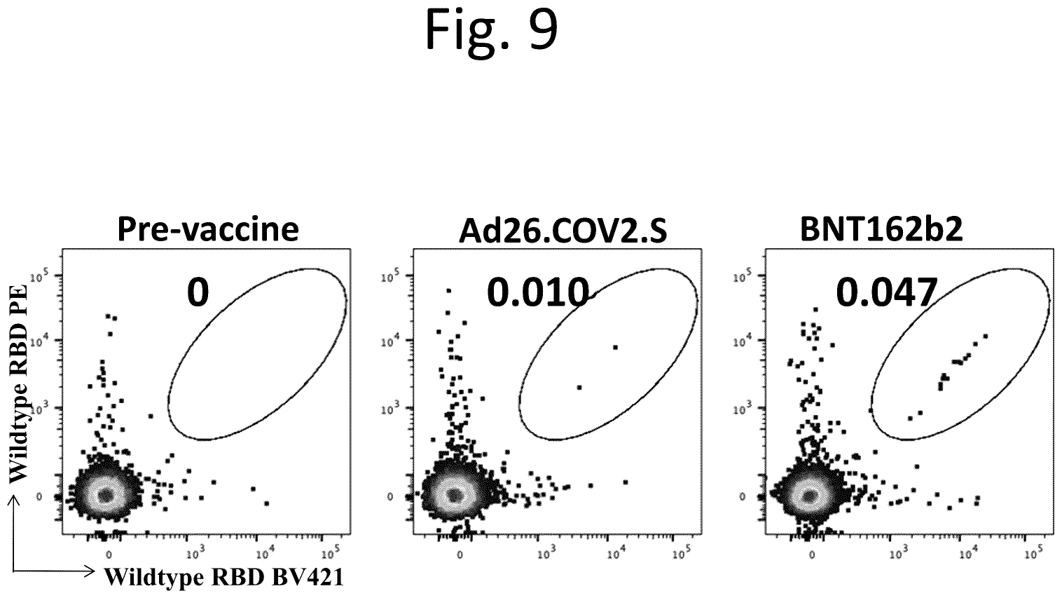

Besides serum antibody, memory B cell level is another key important factor for vaccine induced B cell immunity (Sallusto, F., et al. (2010)). The lower serum antibody level by the adenovirus based vaccines may also correlate with lower memory B cell level as most plasma cells and memory B cells have gone through the same germinal center selection process (Nutt, S. L., et al. (2015)). The data disclosed herein indicate that the percentage of wildtype RBD memory B cells in PBMC after mRNA vaccination is much higher than after adenovirus vaccine (FIG. 9). Double colored mutant RBD tetramer and wildtype RBD with a third color is used to stain more PBMC samples from a biobank (at National Jewish Health) to investigate whether adenovirus based COVID-19 vaccine would also induce mutation specific memory B cells as the mRNA vaccines do together to compare its percentage if this was true.

The biological activity of mutation specific memory B cells after wildtype antigen boost is characterized and directly compared to the percentage of both mutation specific and wildtype RBD memory B cells induced by different types of COVID-19 vaccines. This data will uncover the timeline of appearance of mutation specific B cells in human as well as whether such cells respond to boost using wildtype spike as the immunogen. As both the serum antibody level and memory B cell percentage are lower in adenovirus vaccines, it is expected that the mutation specific B cell level might be also lower in adenovirus vaccines. This may be one major reason for lower protection rate in adenovirus based COVID-19 vaccines compared with mRNA vaccines.

As Ad26.COV2.S is one-dose vaccine (Sadoff, J., et al. (2021)) and due to even lower level of RBD memory cells after adenovirus vaccine, more PBMCs is used as input. Also taken into consideration is that B cell immunity is one branch of immunity induced by vaccines and thus the role of T cell immunity (Tarke, A. et al. SARS-COV-2 vaccination induces immunological T cell memory able to cross-recognize variants from Alpha to Omicron. Cell 185, 847-859, (2022)) when assessing the efficacy of different vaccines is assessed.

### Example 5

This example shows that the wild-type boost does not boost BA5 specific memory B cells. The wildtype spike vaccine boost shot boosts both wildtype specific and cross-reactive memory B cells, but not BA5 mutation specific B cells. This staining is done by mixing four RBD tetramers at the same concentration for staining, two tetramers with wildtype RBD with AF488 and AF647 and two tetramers with Omicron BA5 with PE and BV421. PBMC samples are from same donor before and after wildtype spike vaccine booster. The gating is done as follows: the gating of wildtype RBD binding B cells; binding of BA5 RBD by wildtype RBD binding B cells (this staining divides wildtype binding B cells into BA5 cross reactive B cells and wildtype specific B cells); the gating of BA5 RBD binding B cells; the binding of wildtype RBD by BA5 RBD binding B cells (this staining divides BA5 binding B cells into wildtype cross reactive B cells and BA5 mutation specific B cells). Samples are either from PBMC sample before wildtype vaccine boost, or from same donor after wildtype vaccine boost. The fold change of wildtype binding, BA5 binding, wildtype specific, cross-reactive, and BA5 mutation specific binding B cells after wildtype vaccine booster is provided in Table 1 below.

TABLE 1

| | | | Wild-type | Cross- | BA5 |
|---|---|---|---|---|---|
| Fold change | Wild-type | BA5 | specific | reactive | specific |
| After/before | 3.72 | 3.83 | 3.53 | 4.3 | 1.03 |
| WT boost | | | | | |

The BA5 memory B cells are functional and proliferate after Omicron BA5 virus infection. This staining is done by mixing four RBD tetramers at same concentration for staining, two tetramers with wildtype RBD with AF488 and AF647 and two tetramers with Omicron BA5 with PE and BV421. PBMC samples are from same donor before and after Omicron BA5 virus infection (in September 2022). The gating was done as follows: the gating of wildtype RBD binding B cells; binding of BA5 RBD by wildtype RBD binding B cells (this staining divides wildtype binding B cells into BA5 cross reactive B cells and wildtype specific B cells; the gating of BA5 RBD binding B cells; binding of wildtype RBD by BA5 RBD binding B cells (this staining divides BA5 binding B cells into wildtype cross reactive B cells and BA5 mutation specific B cells). The fold change of wildtype binding, BA5 binding, wildtype specific, cross-reactive, and BA5 mutation specific binding B cells after Omicron BA5 virus infection is provided in Table 2 below.

TABLE 2

| | | | Wild-type | Cross- | BA5 |
|---|---|---|---|---|---|
| Fold change | Wild-type | BA5 | specific | reactive | specific |
| After/before | 2.16 | 2.43 | 2.11 | 2.6 | 1.95 |
| WT boost | | | | | |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### REFERENCES

1 Kaplon, H. & Reichert, J. M. Antibodies to watch in 2021. mAbs 13, 1860476, (2021).
2 Chen, R. E. et al. In vivo monoclonal antibody efficacy against SARS-COV-2 variant strains. Nature 596, 103-108, (2021).
3 Liu, H., Wei, P., Kappler, J. W., Marrack, P. & Zhang, G. SARS-COV-2 Variants of Concern and Variants of Interest Receptor Binding Domain Mutations and Virus Infectivity. Frontiers in Immunology 13, (2022).
4 Polack, F. P. et al. Safety and Efficacy of the BNT162b2 mRNA Covid-19 Vaccine. N. Engl. J. Med. 383, 2603-2615, (2020).
5 Baden, L. R. et al. Efficacy and Safety of the mRNA-1273 SARS-COV-2 Vaccine. N Engl J Med, (2020).
6 Falsey, A. R. et al. Phase 3 Safety and Efficacy of AZD1222 (ChAdOx1 nCOV-19) Covid-19 Vaccine. New England Journal of Medicine, (2021).
7 Gao, Q. et al. Development of an inactivated vaccine candidate for SARS-COV-2. Science 369, 77-81, (2020).
8 Cao, Y. et al. Omicron escapes the majority of existing SARS-COV-2 neutralizing antibodies. Nature 602, 657-663, (2022).
9 Bosshard, H. R. in Peptides (ed Bernd Gutte) 419-454 (Academic Press, 1995).
10 Liu, H. et al. 501Y.V2 and 501Y.V3 variants of SARS-COV-2 lose binding to bamlanivimab in vitro. mAbs 13, 1919285, (2021).
11 Piccoli, L. et al. Mapping Neutralizing and Immunodominant Sites on the SARS-COV-2 Spike Receptor-Binding Domain by Structure-Guided High-Resolution Serology. Cell 183, 1024-1042. (2020).
12 Barnes, C. O. et al. SARS-COV-2 neutralizing antibody structures inform therapeutic strategies. Nature 588, 682-687, (2020).
13 Goel, R. R. et al. Efficient recall of Omicron-reactive B cell memory after a third dose of SARS-COV-2 mRNA vaccine. Cell, (2022).
14 Viant, C. et al. Antibody Affinity Shapes the Choice between Memory and Germinal Center B Cell Fates. Cell 183, 1298-1311. (2020).
15 Purtha, W. E., Tedder, T. F., Johnson, S., Bhattacharya, D. & Diamond, M. S. Memory B cells, but not long-lived plasma cells, possess antigen specificities for viral escape mutants. Journal of Experimental Medicine 208, 2599-2606, (2011).
16 Lan, J. et al. Structure of the SARS-COV-2 spike receptor-binding domain bound to the ACE2 receptor. Nature 581, 215-220, (2020).
17 Sallusto, F., Lanzavecchia, A., Araki, K. & Ahmed, R. From Vaccines to Memory and Back. Immunity 33, 451-463, (2010).
18<COVID-19 Incidence and Death Rates Among Unvaccinated and Fully Vaccinated Adults with and Without Booster Doses During Periods of Delta and Omicron Variant Emergence—25 U.S. Jurisdictions, Apr. 4-Dec. 25, 2021.pdf>.
19 Peck Kayla, M., Lauring Adam, S. & Sullivan Christopher, S. Complexities of Viral Mutation Rates. Journal of Virology 92.
20 Greaney, A. J. et al. Complete Mapping of Mutations to the SARS-COV-2 Spike Receptor-Binding Domain that Escape Antibody Recognition. Cell Host & Microbe 29, 44-57. (2021).
21 Cao, Y. et al. B.1.1.529 escapes the majority of SARS-COV-2 neutralizing antibodies of diverse epitopes. bioRxiv, 2021.2012.2007.470392, (2021).
22 Nutt, S. L., Hodgkin, P. D., Tarlinton, D. M. & Corcoran, L. M. The generation of antibody-secreting plasma cells. Nature Reviews Immunology 15, 160-171, (2015).
23 Nuñez, G., Hockenbery, D., McDonnell, T. J., Sorensen, C. M. & Korsmeyer, S. J. Bcl-2 maintains B cell memory. Nature 353, 71-73, (1991).
24 Lau, A. W. & Brink, R. Selection in the germinal center. Curr Opin Immunol 63, 29-34, (2020).
25 Inoue, T., Shinnakasu, R. & Kurosaki, T. Generation of High Quality Memory B Cells. Frontiers in Immunology 12, (2022).
26 Palm, A.-K. E. & Henry, C. Remembrance of Things Past: Long-Term B Cell Memory After Infection and Vaccination. Frontiers in Immunology 10, (2019).
27 Uriu, K. et al. Neutralization of the SARS-COV-2 Mu Variant by Convalescent and Vaccine Serum. New England Journal of Medicine, (2021).
28 Cameroni, E. et al. Broadly neutralizing antibodies overcome SARS-COV-2 Omicron antigenic shift. Nature 602, 664-670, (2022).
29 Levin, E. G. et al. Waning Immune Humoral Response to BNT162b2 Covid-19 Vaccine over 6 Months. New England Journal of Medicine 385, (2021).
30 Regev-Yochay, G. et al. Efficacy of a Fourth Dose of Covid-19 mRNA Vaccine against Omicron. New England Journal of Medicine, (2022).
31 Lee, I. J. et al. Omicron-specific mRNA vaccine induced potent neutralizing antibody against Omicron but not other SARS-COV-2 variants. bioRxiv, (2022).
32 Di Noia, J. M. & Neuberger, M. S. Molecular Mechanisms of Antibody Somatic Hypermutation. Annual Review of Biochemistry 76, 1-22, doi:10.1146/annurev.biochem.76.061705.090740 (2007).
33 Mesin, L., Ersching, J. & Victora, Gabriel D. Germinal Center B Cell Dynamics. Immunity 45, 471-482, (2016).
34 Starr, T. N., Greaney, A. J., Dingens, A. S. & Bloom, J. D. Complete map of SARS-CoV-2 RBD mutations that escape the monoclonal antibody LY-CoV555 and its cocktail with LY-CoV016. Cell Reports Medicine 2, 100255, (2021).
35 Erijman, A., Rosenthal, E. & Shifman, J. M. How Structure Defines Affinity in Protein-Protein Interactions. PLOS ONE 9, (2014).
36 Jones Bryan, E. et al. The neutralizing antibody, LY-CoV555, protects against SARS-CoV-2 infection in nonhuman primates. Science Translational Medicine 13, (2021).
37 Wang, Z. et al. Naturally enhanced neutralizing breadth against SARS-COV-2 one year after infection. Nature 595, 426-431, (2021).
38 Laidlaw, B. J. & Cyster, J. G. Transcriptional regulation of memory B cell differentiation. Nature Reviews Immunology 21, 209-220, (2021).
39 Liu, H. et al. The basis of a more contagious 501Y.V1 variant of SARS-COV-2. Cell Research 31, 720-722, (2021).
40 Sadoff, J. et al. Safety and Efficacy of Single-Dose Ad26.COV2.S Vaccine against Covid-19. New England Journal of Medicine 384, 2187-2201, doi: 10.1056/NEJMoa2101544 (2021).
41 Heath, P. T. et al. Safety and Efficacy of NVX-CoV2373 Covid-19 Vaccine. New England Journal of Medicine 385, 1172-1183, (2021).
42 Victora, G. D. & Nussenzweig, M. C. Germinal Centers. Annual Review of Immunology 40, 413-442, (2022).
43 Muecksch, F. et al. Increased memory B cell potency and breadth after a SARS-COV-2 mRNA boost. Nature, (2022).
44 Terreri, S. et al. Persistent B cell memory after SARS-COV-2 vaccination is functional during breakthrough infections. Cell Host & Microbe 30, 400-408.e404, (2022).
45 Dashdorj, N. J. et al. Direct comparison of antibody responses to four SARS-COV-2 vaccines in Mongolia. Cell Host & Microbe 29, 1738-1743, (2021).
46 Khoury, D. S. et al. Neutralizing antibody levels are highly predictive of immune protection from symptomatic SARS-COV-2 infection. Nature Medicine 27, 1205-1211, (2021).
47 Liu, H. et al. The Lambda variant of SARS-COV-2 has a better chance than the Delta variant to escape vaccines. bioRxiv, 2021.2008.2025.457692 (2021).
48 Tarke, A. et al. SARS-COV-2 vaccination induces immunological T cell memory able to cross-recognize variants from Alpha to Omicron. Cell 185, 847-859, (2022).

What is claimed is:

1. A method of identifying wild-type specific memory B cells, cross-reactive memory B cells and mutation specific memory B cells in a subject, wherein the subject has been administered one or more doses of a COVID-19 vaccine, the method comprising:
a. contacting a peripheral blood mononuclear cell (PBMC) sample from the subject with an equal concentration of: wild-type SARS-COV-2 RBD tetramers comprising a double colored fluorescent, and a variant SARS-COV-2 RBD tetramer comprising a third color fluorescent label to identify wildtype specific memory B cells and cross-reactive memory B cells; and
b. contacting the PBMC sample with an equal concentration of: variant SARS-COV-2 RBD tetramers comprising a double-colored fluorescent label, and a wild-type RBD tetramer comprising a third color fluorescent label to identify mutation specific memory B cells and cross-reactive memory B cells.

2. A method of determining the efficacy of a vaccine to a viral variant comprising:
a. obtaining a peripheral blood mononuclear cell (PBMC) sample from a subject who has been administered one or more doses of a viral vaccine;
b. contacting the PBMC sample with one or more viral variant receptor binding domain (RBD) tetramers, wherein the tetramers are each labeled with a different fluorescent label, and further contacting the sample with one or more corresponding wild-type RBD tetramers, wherein the wild-type tetramers are labeled with fluorescent labels different from the RBD tetramer labels; and
c. determining binding levels of the wild-type RBD specific, cross-reactive and viral variant RBD specific memory B cells in the PBMC sample from the subject, wherein the levels of cross-reactive and viral variant specific memory B cells correlate with vaccine efficacy against the viral variant.

3. The method of claim 2, wherein the vaccine is a COVID-19 vaccine.

4. The method of claim 2, wherein the viral variant is a SARS-COV-2 variant.

5. The method of claim 2, wherein the vaccine is an influenza vaccine.

6. The method of claim 2, wherein the viral variant is an influenza variant.

7. The method of claim 2, wherein the wild-type RBD tetramer is a wild-type SARS-COV-2 tetramer.

8. The method of claim 2, wherein the variant RBD tetramer is a variant SARS-COV-2 tetramer.

9. The method of claim 2, wherein the step of contacting the PBMC sample from the subject comprises contacting the sample with an equal concentration of wild-type SARS-COV-2 RBD tetramers comprising a double-colored fluorescent label, and a variant SARS-COV-2 RBD tetramer comprising a third color fluorescent label.

10. A method of monitoring a subject's memory B cell response against a vaccine antigen and/or a viral variant thereof, the method comprising:
a. obtaining a PBMC sample from the subject prior to administration of a viral vaccine;
b. obtaining a PBMC sample from the subject following administration of the viral vaccine administered in step a;
c. contacting the PBMC samples from steps a and b with wild-type double colored fluorescent labeled vaccine antigen tetramers and double colored variant antigen tetramers fluorescent labeled with third and fourth colors;
d. detecting binding levels of wild-type specific, cross-reactive and variant antigen specific B cells in the PBMC samples from step c; and
e. comparing the levels of the wild-type specific, cross-reactive and variant antigen specific binding B cells in the sample following administration of the vaccine to the binding levels of memory B cells in the sample prior to administration of the vaccine; wherein percent changes of antigen binding B cell indicates the subject's B cell response to vaccination.

11. The method of claim 10, wherein the vaccine is a COVID-19 vaccine.

12. The method of claim 10, wherein the viral variant is a SARS-COV-2 variant.

13. The method of claim 10, wherein the vaccine is an influenza vaccine.

14. The method of claim 10, wherein the viral variant is an influenza variant.

15. The method of claim 10, wherein the wild-type antigen tetramer is a wild-type SARS-COV-2 antigen tetramer.

16. The method of claim 10, wherein the variant antigen tetramer is a variant SARS-CoV-2 antigen tetramer.

17. A method of restoring therapeutic antibody efficacy against a viral variant, comprising structurally modifying a non-variant/wild-type viral antibody to produce a viral variant specific antibody, the method comprising modifying one or more amino acids in the non-variant/wild-type antibody to be compatible with known mutations in the viral variant; measuring the binding affinity between the non-variant virus antibody and the variant viral protein, measuring the binding affinity between the mutation specific antibody and the variant viral protein, wherein similar binding affinities indicate the viral variant specific antibody has therapeutic efficacy.

18. The method of claim 17, wherein the viral variant is a SARS-COV-2 variant.

19. The method of claim 17, wherein the viral variant is an influenza variant.
